(19) United States
(12) Patent Application Publication
(10) Pub. No.: US 2020/0030486 A1
(43) Pub. Date: Jan. 30, 2020

(54) METHODS OF MAKING FIBRIN COMPOSITIONS AND ARTICLES

(71) Applicant: 3M INNOVATIVE PROPERTIES COMPANY, St. Paul, MN (US)

(72) Inventors: Jonathan J. O'HARE, Oakdale, MN (US); Mikhail L. PEKUROVSKY, Bloomington, MN (US); Amy S. DETERMAN, Mahtomedi, MN (US); Gino L. PITERA, Minneapolis, MN (US); Jason W. BJORK, Cottage Grove, MN (US); Daniel V. NORTON, St. Paul, MN (US); Robert A. ASMUS, Hudson, WI (US)

(21) Appl. No.: 16/338,140

(22) PCT Filed: Oct. 3, 2017
(86) PCT No.: PCT/US2017/054892

§ 371 Date: Mar. 29, 2019

Related U.S. Application Data
(60) Provisional application No. 62/405,111, filed on Oct. 6, 2016.

(51) Int. Cl. A61L 26/00; B29C 43/24

(52) U.S. Cl. CPC A61L 26/0042; A61L 26/008; B29K 2089/00; A61L 2300/412; B29C 43/24

(57) ABSTRACT

A method of forming a fibrin hydrogel composition including providing one or more unitary masses of a fibrin hydrogel, dividing at least one of the unitary masses of the fibrin hydrogel into a multiplicity of smaller pieces of the fibrin hydrogel, and recombining at least a portion of the smaller pieces into a cohesive mass. Dividing at least one of the unitary masses of fibrin hydrogel into a multiplicity of smaller pieces may include shearing or cutting the unitary masses to form an aqueous dispersion of the fibrin hydrogel in an aqueous medium. The aqueous dispersion of fibrin hydrogel may be applied to a substrate on a roller or an endless belt, and is optionally overlaid by a scrim. The cohesive mass of fibrin hydrogel, which may be formed by removing at least a portion of the aqueous medium from the aqueous dispersion of the smaller pieces of the fibrin hydrogel, finds uses in wound dressing articles.

### FIELD

The present disclosure relates generally to wound dressing materials, and more particularly to medical films for the protection of wounds. The disclosed films include fibrin to promote wound closure and healing.

### BACKGROUND

Fibrinogen is cleaved and polymerized into fibrin using thrombin in a well-characterized process. Thrombin cleaves fibrinogen, forming fibrin monomers. Once fibrinogen is cleaved, fibrin monomers come together and form a covalently crosslinked fibrin network in the presence of factors, such as Factor XIII, normally present in blood. At a wound site, the fibrin network helps to close the wound and promote healing.

Various attempts have been made to provide fibrin in a form useful for treating wounds. Perhaps the most commonly known is the in situ generation of fibrin glue, typically performed by delivering separate solutions of fibrinogen and thrombin from a dual-barrel syringe. International Patent Publication No. WO 97/44015 (Heath et al.) describes soluble microparticles including fibrinogen or thrombin, in free-flowing form. It is stated that these microparticles can be mixed to give a dry powder, to be used as a fibrin sealant that is activated only at a wound site. International Patent Publication No. WO 2009/120433 A2 (Delmotte et al.) describes a fibrin material and method for producing the same.

Additionally, various wound cleaning or wound dressing articles containing fibrin have been disclosed. For example, fibrin-containing sponges are disclosed in U.S. Pat. No. 4,442,655. These fibrin-containing sponges have a porous structure and are formed by freeze-drying of a solution containing fibrin partially cross-linked due to the presence of an anticoagulant increasing the clotting time. U.S. Pat. No. 6,599,515 discloses forming a porous structure by lyophilizing a solution containing fibrin partially cross-linked due to the presence of a sufficient amount of a calcium inhibiting or blocking agent. U.S. Pat. Nos. 6,074,663 and 8,529,941 disclose discrete sheets of fibrin-containing material that can be used as wound dressings. U.S. Pat. No. 6,486,377 B2 (Rapp et al.) describes a biodegradable, flexible wound covering based on fibrin and a process for its preparation, in which a fibrinogen solution is subjected to a single-stage or multi-stage dialysis, then a flexible fibrin web is formed by action of a thrombin solution on the fibrinogen solution and this is subsequently subjected to freeze-drying. WO2014/209620 describes fibrin-coated wound dressing articles.

### SUMMARY

The art continually searches for new compositions effective in delivering fibrin to the wound site, and methods of making fibrin-containing wound dressing materials.

Thus, in one aspect, the disclosure describes a fibrin composition including a dehydrated fibrin hydrogel inter-dispersed with 0.5 to 99 wt.-% of a carrier material, wherein the carrier material is not water, a plasticizer, or a mixture thereof. The fibrin composition further includes a salt at a concentration no greater than 20 wt.-%.

In one exemplary embodiment, a fibrin composition is provided, the fibrin composition including a fibrin hydrogel having a fibrin concentration ranging from 0.1 to 15 wt.-%, a carrier material, and a fibrin hydrogel forming salt. The concentration of the carrier material typically ranges from about 0.1 to about 50 wt.-%. The fibrin hydrogel forming salt generally has a concentration less than a threshold concentration to form the fibrin hydrogel. In typical exemplary embodiments, the fibrin hydrogel is at least partially dehydrated.

In further exemplary embodiments, the fibrin composition and dehydrated fibrin hydrogel typically have a salt concentration no greater than 20, 15, 10, or 5 wt.-%. The fibrin hydrogel or fibrin composition can be in various physical forms such a sheet, foam, or plurality of pieces. In certain exemplary embodiments, the carrier material is a polymer. The carrier material may optionally further include a swelling agent and/or a modifying polymer.

In another aspect, a method of forming a fibrin hydrogel composition is described. The method includes providing a composition including a fibrin hydrogel or precursor thereof, and a fibrin hydrogel forming salt. The fibrin hydrogel forming salt concentration is generally greater than or equal to the threshold concentration to form a fibrin hydrogel. The method further includes combining the fibrin hydrogel with a carrier material. The concentration of the carrier material typically ranges from 0.1 to about 50 wt.-%. The method further includes reducing the salt concentration below the threshold concentration to form a fibrin hydrogel. The step of reducing the salt concentration can occur before and/or after combining the fibrin hydrogel with the carrier material.

In some exemplary embodiments, the fibrin hydrogel precursor may be an aqueous solution including fibrinogen, fibrin-forming enzyme, and a fibrin hydrogel forming salt. The salt typically is a calcium salt in combination with other fibrin hydrogel forming salts such as, for example, NaCl. The threshold salt concentration of the aqueous solution is generally at least 0.45 wt.-%, or 0.50 wt.-%, or 0.6 wt.-%, or 0.7 wt.-%, or 0.8 wt.-% or 0.9 wt.-%. In some embodiments, the aqueous solution further includes a fibrin hydrogel plasticizer.

In yet another aspect, a method of forming a fibrin-containing article is described, the method including providing a (e.g., dehydrated) fibrin composition as described herein, and disposing the fibrin composition on or within a substrate such as a release liner, a polymeric film, a polymeric foam, or a nonwoven or woven fibrous material.

Various unexpected results and advantages are obtained in exemplary embodiments of the present disclosure. Thus, in some exemplary embodiments, the disclosed methods may be used to produce semi-continuous rolls of cohesive fibrin-containing gel layers on a substrate in a semi-continuous roll-to-roll process. In further exemplary embodiments, the disclosed methods may produce cohesive fibrin-containing gel layers, either on a substrate, or as a self-supporting film, following removal of the substrate.

Furthermore, in additional exemplary embodiments, the process of forming a fibrin hydrogel composition by forming an aqueous dispersion of the fibrin hydrogel by dividing a unitary mass of the fibrin hydrogel into a plurality of smaller pieces of the fibrin hydrogel, and subsequently recombining at least a portion of the smaller pieces into a cohesive mass, facilitates rapid washing of the fibrin hydrogel to remove the salts and other electrolytes used to form the hydrogel, for example, by exposing fibrinogen to a fibrin-forming salt. We have surprisingly discovered that the processes described herein facilitate rapid and efficient removal of the salt or other electrolytes from the resulting fibrin hydrogels. Such salts and other electrolytes, if not removed from the fibrin gel, have been found to have negative effects on wound healing when the fibrin gel is incorporated into a wound dressing material.

### LISTING OF EXEMPLARY EMBODIMENTS

A. A method of forming a fibrin hydrogel composition comprising

providing one or more unitary masses of a fibrin hydrogel comprising fibrin;

dividing at least one of the unitary masses of the fibrin hydrogel into a plurality of smaller pieces of the fibrin hydrogel;

recombining at least a portion of the smaller pieces into a cohesive mass, optionally wherein the cohesive mass is on a substrate.

B. The method of embodiment A, wherein dividing at least one of the unitary masses of the hydrogel into a plurality of smaller pieces of the fibrin hydrogel comprises shearing the one or more unitary masses of the fibrin hydrogel to form an aqueous dispersion of the smaller pieces of the fibrin hydrogel in an aqueous medium.
C. The method of embodiment B, further comprising adding an aqueous liquid to the one or more unitary masses of the fibrin hydrogel.
D. The method of any one of embodiments A-C wherein the smaller pieces of the fibrin hydrogel exhibit a particle size of from 1 micrometer to no greater than 5 mm, 4 mm, 3 mm, 2 mm, or 1 mm.
E. The method of any one of embodiments C-D, wherein the one or more unitary masses of the fibrin hydrogel comprise a fibrin hydrogel-forming salt, further wherein the fibrin hydrogel-forming salt has a concentration greater than or equal to a threshold concentration required to form a fibrin hydrogel.
F. The method of embodiment E, further comprising reducing the concentration of the fibrin hydrogel-forming salt below the threshold concentration required to form a fibrin hydrogel.
G. The method of any one of embodiments E-F, wherein the fibrin hydrogel-forming salt is a calcium salt.
H. The method of any one of embodiments E-G, wherein the threshold concentration required to form a fibrin hydrogel is at least 0.45 wt-%, or 0.50 wt-%, or 0.6 wt-%, or 0.7 wt-%, or 0.8 wt-% or 0.9 wt-%.
I. The method of any one of embodiments F-H wherein reducing the concentration of the fibrin hydrogel-forming salt below the threshold concentration required to form a fibrin hydrogel comprises rinsing the unitary fibrin hydrogel, the smaller pieces of the fibrin hydrogel, or a combination thereof, with an aqueous rinse solution.
J. The method of embodiment I, further comprising separating the smaller pieces of the fibrin hydrogel from one or more of the aqueous medium, the aqueous liquid, and the aqueous rinse solution.
K. The method of any one of embodiments A-J, further comprising combining the smaller pieces of the fibrin hydrogel comprising fibrin with at least one of a fibrin hydrogel plasticizer, a fibrin hydrogel swelling agent, a water soluble (co)polymer having a Fikentscher K-value of at least K-90, or a combination thereof.
L. The method of embodiment K, wherein the fibrin hydrogel plasticizer comprises a sugar alcohol, an alkane diol, or a combination thereof.
M. The method of embodiment K, wherein the fibrin hydrogel swelling agent comprises glycerol or polyglycerol 3.
N. The method of embodiment K, wherein the water soluble (co)polymer having a Fikentscher K-value of at least K-90 is cross-linked within the cohesive mass, optionally wherein the water soluble (co)polymer having a Fikentscher K-value of at least K-90 is poly(vinyl)pyrollidone.
O. The method of any one of embodiments B-N, further comprising casting the aqueous dispersion of the smaller pieces of the fibrin hydrogel in the aqueous medium on a roller or an endless belt, and removing at least a portion of the aqueous medium from the aqueous dispersion, the fibrin hydrogel, or both the aqueous dispersion and the fibrin hydrogel, to form the cohesive mass.
P. The method of embodiment O, further comprising providing a carrier substrate between the cast aqueous dispersion and the roller or endless belt.
Q. The method of embodiment P, wherein casting the aqueous dispersion of the smaller pieces of the fibrin hydrogel in the aqueous medium on the roller or endless belt produces a continuous coating of the fibrin hydrogel on the carrier substrate, or produces a discontinuous coating of the fibrin hydrogel on the carrier substrate.
R. The method of embodiment Q, wherein the discontinuous coating comprises a plurality of wavy lines, a plurality of parallel lines, a plurality of non-parallel lines, a plurality of dots, or a combination thereof.
S. The method of embodiment Q or R, further comprising providing a carrier layer or scrim on a major surface of the cast aqueous dispersion on the carrier substrate.
T. The method of embodiment S, wherein at least one of the carrier substrate and the carrier layer or scrim is water permeable.
U. The method of any one of embodiments P-T, wherein the carrier substrate comprise a woven or a nonwoven material.
V. The method of embodiment S, wherein removing at least a portion of the aqueous medium from the aqueous dispersion, the fibrin hydrogel, or both the aqueous dispersion and the fibrin hydrogel, comprises applying pressure to the cast aqueous dispersion to form the cohesive mass.
W. The method of embodiment V, wherein applying pressure to the cast aqueous dispersion comprises conveying the cast aqueous dispersion through one or more nip rollers.
X. The method of embodiment V, wherein applying pressure to the cast aqueous dispersion comprises wrapping the cast aqueous dispersion, positioned between the carrier substrate and the carrier layer or scrim, around one or more rollers while maintaining the carrier substrate and the carrier layer or scrim under tension.
Y. The method of embodiment V, wherein applying pressure to the cast aqueous dispersion comprises wrapping the cast aqueous dispersion, positioned between the carrier substrate and the carrier layer or scrim, around a water permeable roller while maintaining the carrier substrate and the carrier layer or scrim under tension.
Z. The method of embodiment V, wherein applying pressure to the cast aqueous dispersion comprises wrapping the cast aqueous dispersion, on one or both of the carrier substrate and the carrier layer or scrim, around a water permeable roller while maintaining an interior portion of the water permeable roller under at a pressure below atmospheric pressure, and while maintaining the carrier substrate and the carrier layer or scrim under tension.
AA. The method of any one of embodiments O-Z, wherein removing at least a portion of the aqueous medium from the aqueous dispersion, the fibrin hydrogel, or both the aqueous dispersion and the fibrin hydrogel, comprises heating the cast aqueous dispersion, freeze-drying the cast aqueous dispersion, vacuum-drying the aqueous dispersion, or combinations thereof.
AB. The method of embodiments S-AA, wherein removing at least a portion of the aqueous medium from the aqueous dispersion, the fibrin hydrogel, or both the aqueous dispersion and the fibrin hydrogel, comprises contacting the substrate on a side opposite the cast aqueous dispersion with an absorbent material, or contacting the cohesive mass, with an absorbent material.
AC. The method of any preceding embodiment, wherein the one or more unitary masses of the fibrin hydrogel is prepared by forming an aqueous mixture comprising fibrinogen, fibrin forming-enzyme, and a fibrin hydrogel-forming salt present at a concentration greater than or equal to a threshold concentration required to form a fibrin hydrogel.
AD. The method of embodiment AC, wherein the fibrinogen is present at a concentration greater than 2 wt.-% of the aqueous mixture, and a continuous film of fibrin hydrogel is formed. AE. The method of embodiment AD, wherein the continuous film of fibrin has a basis weight of 2 to 30 mg/cm².
AF. The method of embodiment AD or AE, wherein the continuous film has a thickness ranging from 10 μm to 200 μm.
AG. The method of any one of embodiments AD-AF, wherein the continuous film has a water content of from 5 to 20 wt.-%.
AH. The method of embodiment AC, wherein the fibrinogen concentration is less than 2 wt.-%, and a discontinuous film of fibrin hydrogel or flakes of fibrin are formed.
AI. The method of any one of embodiments P-AH, further comprising removing the carrier substrate from the cohesive mass.
AJ. The method of any one of embodiments P-AE, wherein the carrier substrate is embedded in the cohesive mass.
AK. The method of any one of embodiments A-AJ wherein the cohesive mass has a salt concentration no greater than 20, 15, 10, or 5 wt-% and a water content no greater than 20 wt.-%.
AL. The method of any one of embodiments A-AK, further comprising dividing the cohesive mass into a plurality of pieces.
AM. The method of any one of embodiments A-AL, wherein the method is a continuous process.
AN. The method of any one of embodiments A-AM, further comprising sterilizing the cohesive mass, optionally using actinic or ionizing irradiation.
AO. A cohesive mass comprising a fibrin hydrogel or a dehydrated fibrin hydrogel prepared by the method of any one of embodiments A-AN.
AP. A wound dressing article comprising the cohesive mass of embodiment AO applied to a substrate, wherein the substrate is selected from a skin adhesive, a release liner, a (co)polymeric film, a (co)polymeric foam, a nonwoven fibrous material, or a woven fibrous material.
AQ. A method of forming a cohesive fibrin gel, comprising:

reacting a solution of lyophilized fibrinogen with thrombin in the presence of a salt to form a fibrin gel containing between about 4 to 6% by weight of fibrin,

washing the fibrin gel to substantially remove the salt,

chopping the fibrin gel so as to form a fibrin gel dispersion in an aqueous medium, wherein the fibrin gel dispersion exhibits a percent solids of from 4 to 20 wt.-% solids,

applying the fibrin gel dispersion to a major surface of a carrier substrate, and

removing at least a portion of the aqueous medium to produce the cohesive fibrin gel.

AR. The method of embodiment AQ, further comprising adding glycerol to the fibrin gel dispersion at a concentration of at least 4 wt.-% based on the weight of the fibrin gel dispersion.
AS. The method of embodiment AQ or AR, further comprising applying a scrim to the fibrin gel dispersion on the major surface of the carrier substrate.
AT. The method of any one of embodiments AQ-AS, further comprising applying pressure to the fibrin gel dispersion on the major surface of the carrier substrate to produce the cohesive fibrin gel.
AU. The method of embodiment AS or AT, further comprising winding the fibrin gel dispersion on the major surface of the carrier substrate and the scrim to form a roll.
AV. The method of any one of embodiments AQ-AT, further comprising removing the scrim from the cohesive fibrin gel on the carrier substrate, and winding the fibrin gel dispersion on the major surface of the carrier substrate to form a roll.
AW. The method of any one of embodiments AQ-AT, further comprising removing the scrim and the carrier substrate from the cohesive fibrin gel.
AX. A wound article, comprising:

a winding core, and

a web comprising a layer comprising a cohesive fibrin gel layer on a major surface of a substrate, wherein the web is wound upon itself in a plurality of 360 degree turns around the winding core.

AY. The wound article of embodiment AX, wherein the substrate is selected from a skin adhesive, a release liner, a (co)polymeric film, a (co)polymeric foam, a nonwoven fibrous material, or a woven fibrous material.

Various aspects and advantages of exemplary embodiments of the disclosure have been summarized. The above Summary is not intended to describe each illustrated embodiment or every implementation of the present certain exemplary embodiments of the present disclosure. The Drawings and the Detailed Description that follow more particularly exemplify certain preferred embodiments using the principles disclosed herein.

### BRIEF DESCRIPTION OF THE DRAWINGS

The disclosure may be more completely understood in consideration of the following detailed description of various embodiments of the disclosure in connection with the accompanying FIGURES, in which:

FIG. 1 is a schematic view of a process for forming a continuous fibrin film.

In the drawings, like reference numerals indicate like elements. While the above-identified drawing, which may not be drawn to scale, sets forth various embodiments of the present disclosure, other embodiments are also contemplated, as noted in the Detailed Description. In all cases, this disclosure describes the presently disclosed disclosure by way of representation of exemplary embodiments and not by express limitations. It should be understood that numerous other modifications and embodiments can be devised by those skilled in the art, which fall within the scope and spirit of this disclosure.

### DETAILED DESCRIPTION

For the following Glossary of defined terms, these definitions shall be applied for the entire application, unless a different definition is provided in the claims or elsewhere in the specification.

### Glossary

Certain terms are used throughout the description and the claims that, while for the most part are well known, may require some explanation. It should understood that:

The term “aqueous” or “aqueous medium” means including water as a constituent.

The terms “(co)polymer” or “(co)polymers” includes homopolymers and copolymers, as well as homopolymers or copolymers that may be formed in a miscible blend, e.g., by coextrusion or by reaction, including, e.g., transesterification. The term “copolymer” includes random, block and star (e.g. dendritic) copolymers.

The term “dispersion” means a mixture exhibiting two or more distinct phases of matter, wherein one phase (the “dispersion medium”) is continuous, and another phase (the “dispersed phsase”) is discontinuous. For example, a fibrin gel dispersion in an aqueous liquid has a discontinuous fibrin gel dispersed phase, and a continuous phase dispersion medium that is an aqueous liquid.

The term “fibrin” refers to a protein formed by the reaction of fibrinogen with a fibrin-forming enzyme (e.g. thrombrin). Such enzyme is capable of cleaving fibrin A and B peptides from fibrinogen and convert it to fibrin. Fibrinogen is a precursor to fibrin.

The term “gel” refers to a dispersion in which the dispersed phase has incorporated at least a portion of the dispersion medium to produce a solid or semi-solid, elastically-deformable material.

The term “homogeneous” means exhibiting only a single phase of matter when observed at a macroscopic scale.

The term “homogenize” means to apply shear to a mixture of materials to form a dispersion.

The term “shear” means to apply a force sufficient to initiate flow of a material.

The term “adjoining” with reference to a particular layer means joined with or attached to another layer, in a position wherein the two layers are either next to (i.e., adjacent to) and directly contacting each other, or contiguous with each other but not in direct contact (i.e., there are one or more additional layers intervening between the layers).

By using terms of orientation such as “atop”, “on”, “over,” “covering”, “uppermost”, “underlying” and the like for the location of various elements in the disclosed coated articles, we refer to the relative position of an element with respect to a horizontally-disposed, upwardly-facing substrate. However, unless otherwise indicated, it is not intended that the substrate or articles should have any particular orientation in space during or after manufacture.

By using the term “overcoated” to describe the position of a layer with respect to a substrate or other element of an article of the present disclosure, we refer to the layer as being atop the substrate or other element, but not necessarily contiguous to either the substrate or the other element.

By using the term “separated by” to describe the position of a layer with respect to other layers, we refer to the layer as being positioned between two other layers but not necessarily contiguous to or adjacent to either layer.

The terms “about” or “approximately” with reference to a numerical value or a shape means +/−five percent of the numerical value or property or characteristic, but expressly includes the exact numerical value. For example, a viscosity of “about” 1 Pa-sec refers to a viscosity from 0.95 to 1.05 Pa-sec, but also expressly includes a viscosity of exactly 1 Pa-sec. Similarly, a perimeter that is “substantially square” is intended to describe a geometric shape having four lateral edges in which each lateral edge has a length which is from 95% to 105% of the length of any other lateral edge, but which also includes a geometric shape in which each lateral edge has exactly the same length.

The term “substantially” with reference to a property or characteristic means that the property or characteristic is exhibited to a greater extent than the opposite of that property or characteristic is exhibited. For example, a substrate that is “substantially” transparent refers to a substrate that transmits more radiation (e.g. visible light) than it fails to transmit (e.g. absorbs and reflects). Thus, a substrate that transmits more than 50% of the visible light incident upon its surface is substantially transparent, but a substrate that transmits 50% or less of the visible light incident upon its surface is not substantially transparent.

As used in this specification and the appended embodiments, the singular forms “a”, “an”, and “the” include plural referents unless the content clearly dictates otherwise. Thus, for example, reference to fine fibers containing “a compound” includes a mixture of two or more compounds. As used in this specification and the appended embodiments, the term “or” is generally employed in its sense including “and/or” unless the content clearly dictates otherwise.

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques. Furthermore, as used in this specification, the recitation of numerical ranges by endpoints includes all numbers subsumed within that range (e.g. 1 to 5 includes 1, 1.5, 2, 2.75, 3, 3.8, 4, and 5).

Various exemplary embodiments of the disclosure will now be described with particular reference to the Drawings. Exemplary embodiments of the present disclosure may take on various modifications and alterations without departing from the spirit and scope of the present disclosure. Accordingly, it is to be understood that the embodiments of the present disclosure are not to be limited to the following described exemplary embodiments, but is to be controlled by the limitations set forth in the claims and any equivalents thereof.

In one aspect, the present disclosure describes a method of forming a fibrin hydrogel composition, including providing one or more unitary masses of a hydrogel comprising fibrin, dividing at least one of the unitary masses of the hydrogel into a multiplicity of smaller pieces of the hydrogel, and recombining at least a portion of the smaller pieces into a cohesive mass. Dividing at least one of the unitary masses of hydrogel into a multiplicity of smaller pieces may include shearing or cutting the unitary masses to form an aqueous dispersion of the fibrin hydrogel in an aqueous medium. Shearing the aqueous dispersion may be achieved using any conventional means known in the art, for example, using low shear mixers, high shear mixers, homogenizers, microfluidizers, and the like.

The aqueous dispersion of fibrin hydrogel may be applied to a substrate, and is optionally overlaid by a scrim. The cohesive mass of fibrin hydrogel, which may be formed by removing at least a portion of the aqueous medium from the aqueous dispersion of the smaller pieces of the fibrin hydrogel, finds uses in wound dressing articles.

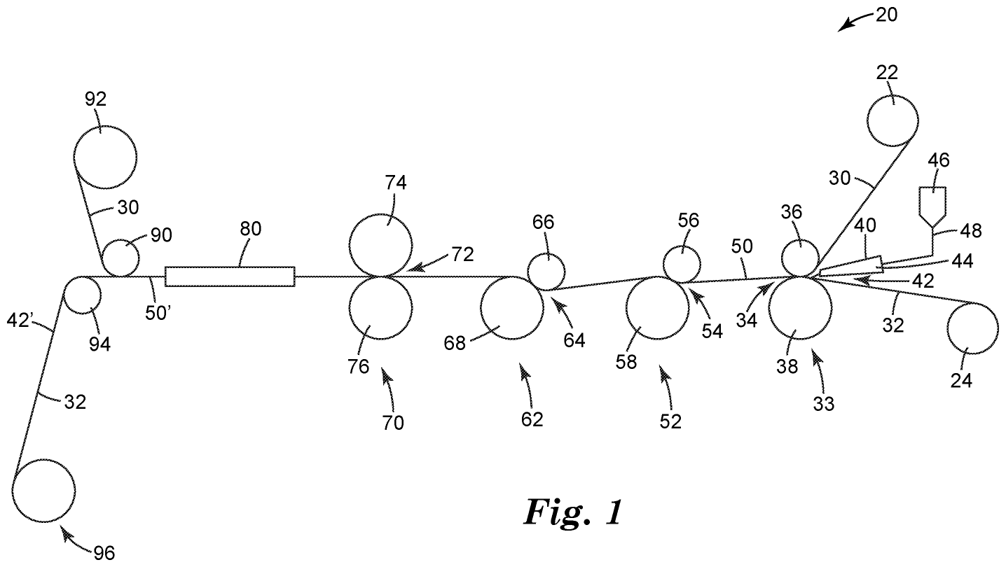

Referring now to FIG. 1, a schematic view of one exemplary process for forming a continuous fibrin film is illustrated. An apparatus 20 includes a first unwind stand 22 and a second unwind stand 24. First unwind stand 22 supplies a scrim 30, while second unwind stand 24 supplies a carrier substrate 32. Scrim 30 and carrier substrate 32 may independently be non-porous or porous, flat or textured. They may be, e.g., foam with open and closed cells, or web that is flat or has a surface structure such as a predetermined roughness, channels, or cells. They may be polymeric film, woven or non-woven fabrics. They may have either low or high surface energy. The presence of scrim 30 as shown in the depicted embodiment is often convenient, but is not considered required by this disclosure.

In some embodiments, it may be convenient for scrim 30 and/or carrier substrate 32 to be a release liner. Various release liners are known such as those made of (e.g. kraft) papers, polyolefin films such as polyethylene and polypropylene, or polyester. The films are preferably coated with release agents such as fluorochemicals or silicones. For example, U.S. Pat. No. 4,472,480 describes low surface energy perfluorochemical liners. Examples of commercially available silicone coated release papers are POLYSLIK™, silicone release papers available from Rexam Release (Bedford Park, Ill.) and silicone release papers supplied by LOPAREX (Willowbrook, Ill.). Other non-limiting examples of such release liners commercially available include siliconized polyethylene terephthalate films commercially available from H. P. Smith Co. and fluoropolymer coated polyester films commercially available from 3M under the brand “ScotchPak™” release liners.

Both scrim 30 and carrier substrate 32 are conveyed to a dispensing station 33 including a nip 34 between a nip roll 36 and backup roll 38. Adjacent to the entrance to nip 34 is a dispensing trough 40 which acts to meter a layer of aqueous dispersion 42 of fibrin gel into nip 34. The sides 44 of dispensing trough 40 act to set the width of the layer of aqueous dispersion 42 entering nip 34. The trough is conveniently feed from dispenser 46 via, e.g., conduit 48. In some alternate embodiments, scrim 30 and carrier substrate 32 may approach dispensing station 33 already laminated together. In these embodiments, a gap may be formed between scrim 30 and carrier substrate 32 partially across the width of these strips and just before nip 34. Then aqueous dispersion 42 may be deposited into that gap.

Besides dispensing via a trough as depicted, the fibrin gel dispersion 42 can be deposited on carrier substrate 32 via other coating methods, including feeding knife coating, die coating, roller coating, screen coating, screen printing, and many other well-known methods. It can be continuous across the width or it can be discontinuous as to form interconnecting and/or disconnected regions. Alternatively, an arrangement of dispensing needles such as disclosed in PCT Pub. App. WO 2016/047343 (“Method and Apparatus for Forming Articles with Non-uniform Discontinuous Patterned Coatings”), U.S. Pat. No. 8,986,786 (“Distribution Manifold with Multiple Dispensing Needles”), U.S. Pat. No. 9,192,960 (“Contact Coating by Use of a Manifold Provided with Capillary Tubes,” or U.S. Pat. No. 9,266,144 (“Method and Apparatus for Producing a Non-uniform Coating on a Substrate”), can be used to lay down oscillating and/or discontinuous patterns of the fibrin gel dispersion.

A multi-layer substrate 50 comprising layer of fibrin gel dispersion 42 sandwiched between scrim 30 and carrier substrate 32 emerges from nip 34 and is conveyed to a first wringing station 52 so that multi-layer substrate 50 can be dewatered. First wringing station 52 includes a nip 54 between wringing roller 56 and backup roller 58. In the depicted embodiment, the now partially de-wetted multi-layer substrate 50 is conveyed to a second wringing station 62 including a nip 64 between a wringing roller 66 and a backup roller 68 to further dewater multi-layer substrate 50. Other expedients for dewatering, such as vacuum belts, vacuum rollers, and the like may be used instead of nip-based stations. Where nips are used, an absorbent web may the conveyed through the nip simultaneously with the multi-layer substrate 50 to increase water removal.

In some alternate embodiments, fibrin gel dispersion 42 may be directly deposited onto a temporary surface such as a belt or a screen and thereafter transferred from that temporary surface onto carrier substrate 32 so as to form multi-layer substrate 50.

The now further de-wetted multi-layer strip 50 is conveyed to a consolidating station 70 comprising a nip 72 between a first consolidating roll 74 and a second consolidating roll 76. While the depicted embodiment shows a separate consolidating station 70, this disclosure teaches that de-watering and consolidation can be achieved in separate or common stations. In the examples below, first and second consolidating rolls 74 and 76 are solid, though resilient rolls. However, in alternate embodiments one or both of these rollers can be impermeable or permeable, e.g., may be a screen. When a screen roller is present, it may apply a partial vacuum to multi-layer strip 50. Consolidating station may comprise more than one nip, or solid and/or perforated belts may replace rollers altogether. Consolidating station 70 may be configured to work to a specified gap or to a specified pressure. Consolidating station 70 may be configured to operate at an ambient or an elevated temperature. In embodiment where the final product will include both the fibrin strip 42′ and the carrier substrate 32, it may be that consolidation is not required in addition to de-watering.

Multi-layer strip 50 is then conveyed to a drying station 80, conveniently in the form of a forced air oven. In alternate embodiments, more or even all of the de-watering can be accomplished by convection or radiant drying. Upon exiting drying station 80, dried multi-layer strip 50′ is ready to be wound into a wound article. In the depicted embodiment, dried multi-layer strip 50′ is conveyed to a stripping roll 90 so that the scrim 30 can be stripped off and directed to weed windup station 92. Carrier substrate 32 supporting a now dried fibrin strip 42′ is directed by idler roller 94 to windup station 96, forming wound article 100. In alternate embodiments, the multi-layer strip 50′ is wound into a wound article while the scrim is still in place. In some alternate embodiments the dried fibrin strip 42′ may be stripped from both scrim 30 and carrier substrate 32 and either wound as a separate web, immediately converted to an alternate form, or transferred onto a different type of substrate with properties useful in the final product or in a subsequent processing operation.

In another aspect, the present disclosure also describes methods of making substrates comprising fibrin compositions. These substrates have the cohesive strength to be processed into wound dressing articles. This allows, for example, the production of wound dressings incorporating the fibrin-containing substrates to be conveniently and cost-effectively prepared in continuous or semi-continuous roller-to-roller processes.

Thus, in one aspect, a method of forming a fibrin hydrogel composition is described. The method comprises forming an aqueous solution comprising fibrinogen, a fibrin-forming enzyme and salt. Thrombin is the most common fibrin-forming enzyme. Alternative fibrin-forming enzymes include batroxobin, crotalase, ancrod, reptilase, gussurobin, recombinant thrombin-like enzymes, as well as venom of 20 to 30 different species of snakes. The fibrin-forming enzyme can be any one or combination of such fibrin-forming enzymes.

Any suitable sources of fibrinogen and thrombin can be used in the preparation of the fibrin hydrogel. For example, the species from which the fibrinogen is obtained could be human, bovine, porcine, or other animal sources. Similarly, thrombin can also be obtained from human, bovine, porcine, or other animal sources. Both fibrinogen and thrombin can also be obtained from recombinant sources. Fibrinogen and thrombin can be obtained commercially as aqueous solutions, and the concentrations of these solutions may vary. Alternatively, fibrinogen and thrombin can be provided in lyophilized form and stored at very low temperatures. Lyophilized fibrinogen is typically reconstituted with sterile water before use. Thrombin is also reconstituted with sterile calcium chloride and water before use. Saline, phosphate buffered solution, or other reconstituting liquid can also be used. In preparing fibrin, the reconstituted fibrinogen and thrombin are then combined to form fibrin.

The aqueous solution generally comprises a sufficient amount of fibrinogen and fibrin-forming enzyme (e.g. thrombin) to produce the desired amount of fibrin. In some embodiments, the amount of fibrinogen in the aqueous solution is at least 1 mg/mL and typically no greater than 120 mg/mL. In some embodiments, the amount of fibrinogen is no greater than 75, 50, 25, 20, 15, 10 or 5 mg/mL. Further, the amount of fibrin-forming enzyme (e.g. thrombin) in the aqueous solution is at least 0.01, 0.02, 0.03, 0.04, or 0.05 Units/milliliter (U/mL) and typically no greater than 500 U/mL. In some embodiments, the amount of fibrin-forming enzyme (e.g. thrombin) in the aqueous solution is no greater than 250, 125, 50, 25, 20, 15, 10, or 5, 4, 3, 2, or 1 U/mL. Aqueous solutions of fibrinogen typically comprise salt (e.g. saline). The salt concentration is sufficient such that the fibrinogen forms a solution. Alternatively, solid fibrinogen can be reconstituted in saline or other salt solution. In a typical embodiment, substantially all the fibrinogen is converted to fibrin. Excess fibrin-forming enzyme (e.g. thrombin) is removed when the fibrin hydrogel is rinsed to reduce the salt content.

The aqueous solution further comprises salt suitable for producing a fibrin containing hydrogel. Thus, such salt can be characterized as a fibrin hydrogel forming salt. The fibrin is generally uniformly dispersed and soluble in the hydrogel. Hence, the hydrogel typically contains little or no fibrin precipitates. When a fibrin hydrogel is formed, the hydrogel is generally a continuous two-phase system that can be handled as a single mass.

Various salts with Group I and/or Group II metal cations have been utilized to solubilize protein such as potassium, sodium, lithium, magnesium, and calcium. Other cations utilized in protein synthesis include ammonium and guanidinium.

Various anions have also been utilized to solubilize protein. Although chloride anion is most common, nitrate and acetate are most similar to chloride according to the Hofmeister series, i.e. a classification of ions in order of their ability to salt out (e.g. precipitate) or salt in (e.g. solubilize) proteins.

In some embodiments, the salt comprises sodium chloride. The amount of sodium chloride in the aqueous solution and fibrin hydrogel, prior to dehydration, is typically greater than 0.09 wt.-% of the solution. The concentration of sodium chloride may be at least 0.10, 0.20, 0.30, 0.04, 0.50, 0.60, 0.70, 0.80 or “normal saline” 0.90 wt.-% and typically no greater than 1 wt.-%. Minimizing the salt concentration is amenable to minimizing the salt that is subsequently removed.

The salt typically comprises a calcium salt, such as calcium chloride. The amount of calcium salt in the aqueous solution and fibrin hydrogel, prior to dehydration, is typically at least 0.0015%, 0.0020%, or 0.0030% wt.-% and typically no greater than 0.5 wt.-%.

In typical embodiments, a buffering agent is also present to maintain the desired pH range. In some embodiments, the pH ranges from 6 to 8 or 7 to 8 during the formation of the fibrin. Various buffering agent are known. Buffering agents are typically weak acids or weak bases. One suitable buffering agent is a zwitterionic compound known as HEPES (4-(2-hydroxyethyl)-1-piperazineethanesulfonic acid). Other buffering agents, such as those commonly known as Good buffers can also be utilized. In some embodiments, the buffering agent does not substantially contribute to the formation of the fibrin hydrogel. For example when the salt contains sodium and calcium chloride, the buffering agent HEPES does not substantially contribute to the formation of the fibrin hydrogel. This means that a fibrin hydrogel can be formed with the sodium and calcium salts in the absence of HEPES. Thus the concentration of HEPES in this example, as well as any other salt that does not substantially contribute to the formation of the fibrin hydrogel, is not included in the threshold concentration to form a fibrin hydrogel.

As depicted in Table 1, of the forthcoming examples when the fibrin hydrogel salt (e.g. NaCl+CaCl₂) concentration was 0.423 wt.-% of the aqueous solution a fibrin hydrogel could not be formed. Without intending to be bound by theory, it is believed that a salt (e.g. NaCl+CaCl₂) concentration of 0.423 wt.-% is insufficient to solubilize the fibrinogen. However, when the concentration of salt was greater than 0.423 wt.-% a fibrin hydrogel readily formed. Hence, the threshold concentration to form a fibrin hydrogel is greater than 0.423 wt.-%. The threshold concentration of salt to form a gel is at least 0.430 wt.-% or 0.440 wt.-%, and in some embodiments at least 0.450, 0.500, 0.550, 0.600, 0.650, 0.700, 0.750, 0.800, 0.850, or 0.900 wt.-% of the aqueous solution. It is appreciated that the threshold concentration may vary to some extent depending on the selection of salt(s). The concentration of salt in the (i.e. initially formed) hydrogel is the same as the concentration of salt in the aqueous solution.

When a fibrin hydrogel is formed using a threshold concentration of salt and the hydrogel is dehydrated, the resulting dehydrated fibrin hydrogel has an even greater concentration of salt. For example as depicted in Table 1 of the forthcoming examples, the fibrin hydrogel forming salt (e.g. NaCl+CaCl₂)) concentration is greater than 10, 15, 20, 25, or 30 wt.-%. As described in further detail in the forthcoming examples, high salt concentrations can cause (e.g. dermal) tissue irritation and damage during the healing process as indicated by inflammatory cell infiltration as well as collagen degeneration and mineralization.

The present method of preparing a fibrin composition comprises forming a fibrin hydrogel from an aqueous composition as previously described, and reducing the salt concentration below the threshold salt concentration to form a fibrin hydrogel. For embodiments wherein the (e.g. dehydrated) fibrin hydrogel is utilized for wound healing, the method comprises reducing the salt concentration below the concentration that can cause (e.g. dermal) tissue irritation and damage during the healing process.

In typical embodiments, the step of reducing the salt concentration comprises rinsing the fibrin hydrogel with a solution capable of dissolving the salt. The solution is typically aqueous comprising at least 50, 55, 60, 65, 70, 75, 80, 85, 90, or 95 wt-%, or greater by volume water. The rinsing solution may further contain other water miscible liquids such as plasticizers. The fibrin hydrogel is typically rinsed with a volume of solution at least 1, 2, 3, 4, 5, 6, 7, 8, 9, or 10 times greater than the volume of the hydrogel. To reduce the salt concentration even further, the fibrin hydrogel may be rinsed with a volume of solution 11, 12, 13, 14, 15, 16, 17, 18, 19, or 20 times greater than the volume of the hydrogel. Another way of reducing the salt includes reacting the cation and/or anion of the salt, or in other words complexing the salt, such that the salt no longer forms ions in an aqueous solution such as bodily fluids of wounds. Another way of reducing the salt concentration is diluting with plasticizer. Further, various combination of these methods can be used.

The amount of fibrin hydrogel forming salt (e.g. NaCl+CaCl₂)) removed from the fibrin hydrogel can depend on the amount of salt in the aqueous (e.g. starting) solution and thus, the amount of salt in the initially formed hydrogel. For example, when the aqueous (e.g. starting) solution comprises about 0.9 wt.-% salt, at least about 35 wt.-% of the salt is removed from the fibrin hydrogel. However, when the aqueous (e.g. starting) solution comprises about 1.25 wt.-% salt, greater than 50% of the salt is removed from the fibrin hydrogel. In some embodiments, at least 40%, 45%, 50%, 55%, 60%, 65%, 70%, 75%, 80%, 85% or 90% of the salt is removed from the hydrogel. In other embodiments, at least 91, 92, 93, 94, 95, 96, 97, 98, or 99% of the salt is removed from the hydrogel. If the threshold concentration is less than 0.9 wt-%, the amount of salt removed can be less than 35 wt.-%. In such embodiment, at least 5%, 10%, 15%, 20%, 25%, 30%, 35%, 40%, or 45% of the salt is removed from the hydrogel.

The fibrin hydrogel having the reduced fibrin hydrogel forming salt content is then dehydrated using any number of methods. This step may be referred to as dehydrating, drying or desiccating the hydrogel, all of which refer herein to the process of removing water content from the hydrogel as possible. Dehydration can therefore be accomplished using heat, vacuum, lyophilization, desiccation, and the like. In some embodiments, lyophilization may be preferred since the resulting fibrin material is less likely to swell once in contact with an aqueous solution. The dehydration step may occur over a range of time, depending on the particular method used and the volume of the hydrogel. For example, the step may last for a few minutes, a few hours, or a few days. The present disclosure is not intended to be limited in this regard.

The dehydrated fibrin hydrogel generally has a hydrogel forming salt concentration less than 30 wt.-% or 25 wt.-% for a water content no greater than 20 wt.-%. When the dehydrated fibrin hydrogel is intended for use for wound healing the salt concentration is less than 20, 19, 18, 17, 16, 15, 14, 13, 12, 11, 10, 9, 8, 7, 6, 5, 4, 3, 2, or 1 wt.-%, or less of the dehydrated fibrin hydrogel having a water content no greater than 20 wt.-%. In some embodiments, the dehydrated hydrogel has a water content no greater than 19, 18, 17, 16, 15, 14, 13, 12, 11, 10, 9, 8, 7, 6, 5, 4, 3, 2, or 1 wt.-% or less. In some embodiments, the total salt concentration including the buffering salts are also within the concentration ranges just described. In some embodiments, the dehydrated hydrogel will swell when combined with water (i.e. rehydrated).

The dehydrated fibrin hydrogel typically has a water content of at least 1, 2, 3, 4, or 5 wt-%. In some embodiments, the dehydrated fibrin hydrogel has a water content of at least about 10, 15, or 20 wt-%.

The fibrin hydrogel is dehydrated to reduce the water content and thereby increase the fibrin concentration. Higher fibrin concentrations generally promote healing more rapidly than lower fibrin concentrations. The fibrin hydrogel, prior to dehydration typically comprises about 0.5 wt.-% to 5 wt.-% fibrin. After dehydration, the fibrin composition typically comprises at least 10, 15, 20, 25, 30, 35, 40, 45, or 50 wt.-% fibrin. The fibrin concentration of the dehydrated hydrogel is typically no greater than 99 wt.-% and in some embodiments no greater than 95, 90, 85, or 80 wt.-%.

Since only a small concentration of fibrin-forming enzyme (e.g. thrombin) is needed to form fibrin and excess fibrin-forming enzyme (e.g. thrombin) is removed during rinsing, the concentration of fibrin-forming enzyme (e.g. thrombin) is also low in the dehydrated fibrin hydrogel. The dehydrated fibrin hydrogel typically includes fibrin-forming enzyme (e.g. thrombin) in an amount of thrombin no greater than 0.05 U/mg, or 0.005 U/mg, or 0.0005 U/mg, or 0.00005 U/mg. In some embodiments, the amount of fibrin-forming enzyme (e.g. thrombin) is 1 or 0.1 ppm relative to the concentration of fibrin.

The (e.g. dehydrated) fibrin hydrogel may include an amount of fibrinogen in a range from 0.1 wt.-% to 10 or 15 wt.-% relative to a total weight of the (e.g. dehydrated) fibrin hydrogel, or any amount within that range. In some embodiments, (e.g. dehydrated) fibrin hydrogel includes fibrinogen in an amount no greater than 5, 4, 3, 2, 1, 0.1 or 0.05 wt.-%, relative to a total weight of the (e.g. dehydrated) fibrin hydrogel. When the conversion of fibrinogen to fibrin is 100%, the dehydrated fibrin hydrogel is substantially free of fibrinogen.

In some embodiments, the fibrin hydrogel further comprises a plasticizer. Various water-miscible plasticizers are suitable for hydrogels. Such plasticizers typically comprise hydroxyl groups. Suitable plasticizers include for example C₃-C₂₄ sugar alcohols such as glycerol, diglycerol, triglycerol, xylitol, and mannitol as well as C₃-C₂₄ alkane diols such as butane diol and propane diol. In some embodiments, the plasticizer comprises an alkylene group having no greater than 12 carbons atoms. The (e.g. dehydrated) fibrin hydrogel may contain a single plasticizer or combination of plasticizers. When plasticizer is present, the concentration typically ranges from 0.5 wt.-% to 2 wt.-% of the aqueous starting solution. The dehydrated hydrogel may comprise at least 5, 10, 15 or 20 wt.-% and typically no greater than 80, 70, 60, 50, or 40 wt-% plasticizer.

Inclusion of a plasticizer can result in a flexible dehydrated hydrogel composition, the properties of which can be determined by standard tensile and elongation testing. The film of flexible dehydrated hydrogel for testing can have a thickness of at least 10, 15 or 20 microns and typically no greater than 2 mm, 1 mm, 500 microns, or 250 microns. In some embodiments, the thickness is no greater than 200, 150, 100, 75, or 60 microns. The elongation can range from 10, 15, or 20% to 1000%. In some embodiments, the elongation (e.g. of a 50 micron film) is at least 50% or 75% and no greater than 200%, 150%, or 100%. The ultimate tensile strength is typically at least 0.1, 0.2, or 0.3 MPa and is typically no greater than 150 MPa. In some embodiments, the ultimate tensile strength (e.g. of a 50 micron film) is no greater than 50, 25, 10, or 5 MPa. The Young's elastic modulus is typically at least 0.5, 0.6, 0.7, 0.8, 0.9 or 1 MPa and is typically no greater than about 2000 MPa. In some embodiments, the Young's elastic modulus (e.g. of a 50 micron film) is at least 2 or 3 MPa and typically no greater than 100, 75 or 50 Mpa.

The (e.g. dehydrated) fibrin hydrogel can include various additives, provided the additives do not detract from forming the fibrin hydrogel and reducing the salt concentration therefrom. Examples of additives can include any of antimicrobial agents, anti-inflammatory agents, topical anesthetics (e.g., lidocaine), other drugs, growth factors, polysaccharides, glycosaminoglycans. If an additive is included, it should be included at a level that does not interfere with the activity of the fibrin containing layer with respect to promoting healing of the wound.

Antimicrobial agents are agents that inhibit the growth of or kill microbes such as bacteria, mycobacteria, viruses, fungi, and parasites. Anti-microbial agents therefore include anti-bacterial agents, anti-mycobacterial agents, anti-viral agents, anti-fungal agents, and anti-parasite agents. Fibrin containing layers so loaded can be used to prevent or control infection.

Anti-inflammatory agents are agents that reduce or eliminate inflammation. Examples include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cin alone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, and zomepirac sodium.

The (e.g. dehydrated) fibrin hydrogel can have various physical forms. In some embodiments, the fibrin hydrogel is formed prior to reducing the salt content. The fibrin hydrogel is typically sufficiently flowable at a temperature ranging from 0° C. to 37° C. such that the fibrin hydrogel takes the physical form of the container surrounding the fibrin hydrogel. For, example if the fibrin hydrogel is cast into a rectangular pan, the fibrin hydrogel forms into a sheet. Thus, the fibrin hydrogel can be cast into various shaped containers or in other words molded to provide (e.g. dehydrated) hydrogel of various shapes and sizes.

In one embodiment, the (e.g. dehydrated) fibrin hydrogel may be provided as a fibrin foam. This can be accomplished by aerating the fibrinogen solution prior to addition of thrombin or aerating the fibrin hydrogel early in the polymerization process. After formation of the fibrin foam, salts can then be removed as previously described.

In another embodiment, the (e.g. dehydrated) fibrin hydrogel may be provided as particles. For example, (e.g. dehydrated) fibrin hydrogel microbeads may be formed, such as by the method described in U.S. Pat. No. 6,552,172 (Marx et al.). In yet another example, (e.g. dehydrated) fibrin hydrogel particles may be utilized as microcarriers such as described in US 2010/0291219 (Karp et al.). The salt content of the microbeads and microcarriers is reduced below the threshold concentration to form a fibrin hydrogel as previously described.

In other embodiments, the dehydrated fibrin hydrogel can be formed after reducing the salt content. For example, a sheet of (e.g. dehydrated) fibrin hydrogel can be (e.g. laser or die) cut into pieces having various shapes and sizes. In another example, the dehydrated hydrogel may be ground, pulverized, milled, crushed, granulated, pounded, and the like, to produce fibrin powder as described in WO2014/209620. In this embodiment, methods used for making (e.g. dehydrated) fibrin hydrogel particles are not dependent on oil-in-water emulsions.

When (e.g. dehydrated) fibrin particles are formed, the method may further involve size separating the particles. This may be accomplished most easily by sieving the particle composition through one or more appropriate sieves or filters having desired pore sizes. In some embodiments the particles can be sieved to arrive at populations having average diameters in the range of about 85-180, 90-170, 100-160, 100-150, 110-150, 120-140, or about 130 micrometers in average diameter. The fibrin particles may be equal to or less than 80, 90, 100, 110, 120, 130, 140, 150, 160, 170, or 180 micrometers, provided they have a minimum average diameter of at least 10, 20, 30, 40 or 50 micrometers. It is to be understood that these average diameters refer to the diameter of the dehydrated particles rather than their rehydrated diameters. The particle volume may increase 10-250% of the initial volume after rehydration.

In some embodiments, fibrin particles can be size restricted. In some aspects, the composition comprises a plurality of fibrin particles, wherein at least 50% of which have an average diameter of 85-180 micrometers prior to hydration. In some embodiments, at least 55%, at least 60%, at least 65%, at least 70%, at least 75%, at least 80%, at least 85%, at least 90%, at least 95%, or more of the fibrin particles have an average diameter of 85-180 micrometers.

The fibrin particles may have spherical shape or an irregular non-spherical shape and size. The diameter of a non-spherical particle can be determined by summing its longest and its shortest dimension and dividing that sum by two. This is referred to as the average diameter of a single particle. Average diameter of a population of particles may be deduced based on a sieving analysis (i.e., the sieving analysis would provide a range of average diameters based on retention and/or flow through of particles). It will be understood that the term “average diameter” of a population of particles, defined as “summing its longest and its shortest dimension and dividing that sum by two”, is conceptually similar to the term “average particle size”, which refers to the “largest dimension” of the particles in a population of the particles.

In some embodiments, fibrin particles are provided that are defined by their surface topology, topography, or roughness. The surface topology or roughness may be expressed in terms of the number and/or size of features (or protrusions) on the surface of the particles. Roughness can be observed using techniques commonly used in the art including optical profilometry and atomic force microscopy. The number of features on these particles may range from 2-100 typically. The size of these features (or protrusions) may be expressed in terms of absolute length or in terms of the ratio of the size of the feature (or protrusion) and the average diameter of the particles. In some embodiments, the size of the feature is about 1 micrometer, about 2 micrometers, about 3 micrometers, about 4 micrometers, about 5 micrometers, about 6 micrometers, about 7 micrometers, about 8 micrometers, about 9 micrometers, about 10 micrometers, or more. In other embodiments, the size of the feature is more than 10 micrometers, more than 15 micrometers, more than 20 micrometers, more than 25 micrometers, more than 30 micrometers, more than 35 micrometers, more than 40 micrometers, more than 45 micrometers, more than 50 micrometers, or more. In still other embodiments, the size is 10-100 micrometers. In other embodiments, the size is 1-10 micrometers. The ratio of feature size and particle average diameter may be about 5%, 10%, 15%, 20%, 25%, 30%, 35%, 40%, 45%, or 50%, or more. This surface roughness is important since it has been found that cells such as connective tissue progenitor cells are better able to bind to particles having a greater degree of surface roughness.

In some embodiments, the (e.g. dehydrated) fibrin hydrogel particles have an average particle size in a range of 0.1 microns up to 100 microns. The fibrin particles can have an average particle size, of at least 0.1, 1, 2, 5, or 10 microns. The average particle is typically no greater than 1000 micrometers, 500, 200 or 100 microns.

The (e.g. dehydrated) fibrin composition described herein may be utilized in the treatment of a wound. To facilitate delivery of the fibrin composition, the fibrin composition (e.g. particles) may be incorporated into a suitable carrier material to form various fibrin-containing gels, pastes, lotions, creams, and ointments. In another embodiment, (e.g. dehydrated) fibrin hydrogel particles can be dispersed in a (e.g. aqueous) liquid carrier material (e.g. an emulsion) to form a fibrin-containing spray.

In other embodiments, (e.g. dehydrated) fibrin particles can be admixed with natural or chemically modified and synthetic biological carrier materials such as collagen, keratin, gelatin, carbohydrates, and cellulose derivatives. Synthetic biological carrier materials can also be utilized such as described in previously cited US 2010/0291219 (Karp et al.).

In some embodiments, the biological carrier material comprises a bioerodible hydrogel such as polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

In other embodiments, the biological carrier material is a biodegradable synthetic polymer such as polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polymers of lactic acid and glycolic acid, polyanhydrides, poly(ortho)esters, polyurethanes, poly(butic acid), poly(valeric acid), and poly(lactide-cocaprolactone) and polyvinylpyrrolidone.

In yet another embodiment, fibrin particles as described herein can be admixed with various (e.g. acrylic or silicone) skin adhesives to form a fibrin-containing skin adhesives.

In typical embodiments, (e.g. dehydrated) fibrin hydrogel particles are provided on or within a substrate at a coating weight that is sufficient to provide the desired effect (e.g. promoting wound re-epithelialization). In some embodiments, the coating weight of the (e.g. dehydrated) fibrin hydrogel particles is typically at least 0.2, 0.5 or 1 milligram per cm² and typically no greater than 20, 10 or 5 milligrams per cm².

The (e.g. dehydrated) fibrin hydrogel composition described herein may be utilized as a wound dressing article. The wound dressing article described herein comprises a (e.g. dehydrated) fibrin composition in a suitable physical form such as a sheet (i.e. film), foam sheet, or fibrin particles disposed on or within a substrate. Thus, the (e.g. dehydrated) fibrin hydrogel layer can be provided in various forms as a continuous or discontinuous layer.

In some embodiments, the substrate of the wound dressing is a flexible film layer, (also referred to as a “backing” layer), typically includes a liquid impervious, moisture vapor permeable (e.g. breatheable) polymeric film. The liquid impervious, moisture vapor permeable polymeric film is a conformable organic polymeric material that preferably retains its structural integrity in a moist environment. Herein, “conformable” films are those that conform to a surface, even upon movement of the surface, as with the surface of a body part. As such, when the flexible film layer is applied to an anatomical feature, it conforms to the surface even when the surface is moved. The preferred flexible film layer is also conformable to animal anatomical joints. When the joint is flexed and returned to its unflexed position, the flexible film layer stretches enough to accommodate the flexion of the joint, but is resilient enough to continue to conform to the joint when the joint is returned to its unflexed condition. A description of this characteristic of flexible film layers preferred for use in wound dressings of the present disclosure can be found, for example, in U.S. Pat. No. 5,088,483 (Heineke) and U.S. Pat. No. 5,160,315 (Heineke).

Suitable films have a composition and thickness that allow for the passage of moisture vapor through them. The film aids in the regulation of water vapor loss from the wound area beneath the dressing. The film also acts as a barrier to both bacteria and to liquid water or other liquids.

The moisture vapor permeable polymeric films for use as flexible film layers in the present disclosure can be of a wide range of thicknesses. In some embodiments, the flexible film layers have a thickness of at least 10 or 12 microns ranging up to 250 microns. In some embodiments, the flexible film layer has a thickness no greater than 75 microns.

Moisture vapor transmission rate (“MVTR”) properties of a wound dressing article are important to allow the wound under the wound dressing to heal in moist conditions without causing the skin surrounding the wound to become macerated, and to facilitate optimum wear time and ease of removal.

A dry MVTR (or upright MVTR) of wound dressings or various components thereof, including the flexible film layer, can be measured by ASTM E-96-80 (American Society of Testing Materials) at 40° C. and 20% relative humidity using an upright cup method. Wet MVTR (or inverted MVTR) can be measured by the same method except that the sample jars are inverted so the water is in direct contact with the test sample.

In some embodiments, the film has a dry MVTR that is less than the wet MVTR of the film. For example, the film may have a dry MVTR of at least 300 g/m²/24 hours and a wet MVTR of at least 500, 1000, 2000 or 3000 g/m²/24 hours. In some embodiments, the film has a wet MVTR greater 10,000 g/m²/24 hours or 15,000 g/m²/24 hours.

Examples of suitable materials for the liquid-impervious, moisture-vapor permeable polymeric films of the flexible film layer include synthetic organic polymers including, but not limited to: polyurethanes commercially available from B.F. Goodrich, Cleveland, Ohio, under the trade designation ESTANE, including ESTANE 58237 and ESTANE 58245; polyetheramide block copolymers commercially available from Elf Atochem, Philadelphia, Pa., under the trade designation PEBAX, including PEBAX MV 1074; polyether-ester block copolymers commercially available from DuPont, Wilmington, Del., under the trade designation HYTREL; and thermoplastic elastomers commercially available from DSM Engineering Plastics, Evansville, Ind., under the trade designation ARNITEL VT. The polymeric films can be made of one or more types of monomers (e.g., copolymers) or mixtures (e.g., blends) of polymers. Preferred materials are thermoplastic polymers, e.g., polymers that soften when exposed to heat and return to their original condition when cooled. A particularly preferred material is a thermoplastic polyurethane.

Flexible films of the wound dressing articles of the present disclosure can also include other breathable materials including, for example, nonwoven, woven, and knit webs, porous films (e.g., provided by perforations or microporous structure), foams, paper, or other known flexible films. A preferred flexible film includes a combination of a liquid-impervious, moisture-vapor permeable polymeric film and a moisture-vapor permeable nonwoven web that can, among other advantages, impart enhanced structural integrity and improved aesthetics to the dressings. These layers of film and web may or may not be coextensive. A preferred such nonwoven web is a melt processed polyurethane (such as that available under the trade designation MORTHANE PS-440 from Morton International, Seabrook, N.H.), or hydroentangled nonwoven polyester or rayon-polyester webs (such as those available under the trade designation SONTARA 8010 or SONTARA 8411 from DuPont, Wilmington, Del.).

In some embodiments, flexible film layer is translucent, semi-transparent, or transparent, although this is not a requirement. Some examples of wound dressings that include a transparent or translucent flexible film layer are available under the trade designation TEGADERM, available from 3M Co., St. Paul, Minn.

A low adhesion coating (low adhesion backsize or LAB) can be provided on the flexible film layer on the side that may come into contact with an optional support layer. The low adhesion coating reduces the need to change the dressing due to unwanted dressing removal when other tapes or devices are placed on the dressing and removed, and reduces the surface friction of the dressing on linen or other fabrics, thereby offering additional protection against the accidental removal of dressing. A description of a low adhesion coating material suitable for use with a wound dressing article of the present disclosure can be found in U.S. Pat. No. 5,531,855 (Heineke) and U.S. Pat. No. 6,264,976 (Heineke).

In some embodiments, the wound dressing comprises an absorbent layer. In some embodiments, the absorbent layer can include an absorbent foam layer, or at least a portion of an absorbent foam layer disposed on the flexible film layer. A suitable foam layer can include, for example, an open cell foam selected from among the open cell foams described in U.S. Pat. No. 6,548,727 (Swenson). Suitable open cell foams preferably have an average cell size (typically, the longest dimension of a cell, such as the diameter) of at least about 30 microns, more preferably at least about 50 microns, and preferably no greater than about 800 microns, more preferably no greater than about 500 microns, as measured by scanning electron microscopy (SEM) or light microscopy. Such open cell foams when used in wound dressings of the present disclosure allow transport of fluid and cellular debris into and within the foam. In some embodiments, the foam includes a synthetic polymer that is adapted to form a conformable open cell foam that absorbs wound exudate. Examples of suitable materials for the absorbent, substantially nonswellable foams include synthetic organic polymers including, but not limited to: polyurethanes, carboxylated butadiene-styrene rubbers, polyesters, and polyacrylates. The polymeric foams can be made of one or more types of monomers (e.g., copolymers) or mixtures (e.g., blends) of polymers. Preferred foam materials are polyurethanes. A particularly preferred foam is a polyurethane, available under the trade designation POLYCRIL 400 from Fulflex, Inc., Middleton, R.I. In other embodiments, the foam comprises or consists of the (e.g. dehydrated) fibrin hydrogel.

In another embodiment, the absorbent layer may comprise a non-woven or a fiber material. In an embodiment where the absorbent material includes a fiber material, the fiber material can be a sheath-core fiber having a central core of absorbent fiber and a sheath comprising pressure-sensitive adhesive.

In some embodiments, the absorbent layer may extend around a peripheral region of the wound dressing, to absorb fluids that might otherwise accumulate on skin and result in undesirable skin degradation (e.g., maceration). In such embodiments, an absorbent layer would not need to be included in a more central region of the wound dressing (e.g., the portion of the wound dressing that is in contact with the wound, or positioned over the wound).

The fibrin article, suitable for use as a wound dressing, may comprise various adhesives to bond layers of the article. The fibrin article may also comprises various PSAs for bonding the article to skin. The (e.g. PSA) adhesive layer can be continuous, discontinuous, pattern coated, or melt-blown, for example.

PSAs typically have a storage modulus (G′) of less than 1×10⁶ dynes/cm² at 25° C. and a frequency of 1 hertz. In some embodiments, the PSA has storage modulus (G′) of less than 9, 8, 7, 6, 5, 4, or 3×10⁵ dynes/cm² at 25° C. and a frequency of 1 hertz.

Examples of PSAs include rubber based adhesives (e.g., tackified natural rubbers, synthetic rubbers, and styrene block copolymers), acrylics (e.g., polymerized (meth)acrylates), poly(alpha-olefins), polyurethanes, and silicones. Amine containing polymers can also be used which have amine groups in the backbone, pendant thereof, or combinations thereof. A suitable example includes a poly(ethyleneimine).

Useful adhesives can be any of those that are compatible with skin and useful for wound dressings, such as those disclosed in U.S. Pat. No. Re. 24,906 (Ulrich), U.S. Pat. No. 5,849,325 (Heinecke et al.), and U.S. Pat. No. 4,871,812 (Lucast et. al.) (water-based and solvent-based adhesives); U.S. Pat. No. 4,833,179 (Young et al.) (hot-melt adhesives); U.S. Pat. No. 5,908,693 (Delgado et al.) (microsphere adhesives); U.S. Pat. Nos. 6,171,985 and 6,083,856 (both to Joseph et al.) (low trauma fibrous adhesives); and, U.S. Pat. No. 6,198,016 (Lucast et al.), U.S. Pat. No. 6,518,343 (Lucast et al.), and U.S. Pat. No. 6,441,082 (Gieselman) (wet-skin adhesives).

Inclusion of medicaments or antimicrobial agents in the adhesive is also contemplated, as described in U.S. Pat. No. 4,310,509 (Berglund) and U.S. Pat. No. 4,323,557 (Rosso).

The adhesive can be coated on the substrate by a variety of processes, including, direct coating, lamination, and hot lamination. In some embodiments, the adhesive may be coated as a microstructured adhesive layer.

Silicone and acrylic based pressure sensitive adhesives are most commonly utilized for adhering to the skin, whereas the other classes of adhesives can be utilized to bond layers of the fibrin article suitable for use as a wound dressing.

Silicone PSAs include two major components, a polymer or gum, and a tackifying resin. The polymer is typically a high molecular weight polydimethylsiloxane or polydimethyldiphenyl-siloxane, that contains residual silanol functionality (SiOH) on the ends of the polymer chain, or a block copolymer including polydiorganosiloxane soft segments and urea terminated hard segments. The tackifying resin is generally a three-dimensional silicate structure that is endcapped with trimethylsiloxy groups (OSiMe₃) and also contains some residual silanol functionality. Examples of tackifying resins include SR 545, from General Electric Co., Silicone Resins Division, Waterford, N.Y., and MQD-32-2 from Shin-Etsu Silicones of America, Inc., Torrance, Calif. Manufacture of typical silicone PSAs is described in U.S. Pat. No. 2,736,721 (Dexter). Manufacture of silicone urea block copolymer PSA is described in U.S. Pat. No. 5,214,119 (Leir et al.).

In some embodiments, the silicone adhesive may be characterized as gentle to skin such as descrived in US2011/0212325, US2011/0206924, US2011/0206923, US2013/0040073, U.S. Pat. Nos. 7,407,709 and 787,268.

In some embodiments, the PSAs is an acrylic PSAs typically having a glass transition temperature of about −20° C. or less and may include from 100 to 60 weight percent of a C4-C12 alkyl ester component such as, for example, various (meth)acrylate monomers including isooctyl acrylate, 2-ethyl-hexyl acrylate and n-butyl acrylate and from 0 to 40 weight percent of a polar component such as, for example, acrylic acid, methacrylic acid, ethylene, vinyl acetate, N-vinyl pyrrolidone and styrene macromer.

Suitable acidic monomers for preparing (meth)acrylic PSAs include those containing carboxylic acid functionality such as acrylic acid, methacrylic acid, itaconic acid, and the like; those containing sulfonic acid functionality such as 2-sulfoethyl methacrylate; and those containing phosphonic acid functionality. Preferred acidic monomers include acrylic acid and methacrylic acid.

Additional useful acidic monomers include, but are not limited to, those selected from ethylenically unsaturated carboxylic acids, ethylenically unsaturated sulfonic acids, ethylenically unsaturated phosphonic acids, and mixtures thereof. Examples of such compounds include those selected from acrylic acid, methacrylic acid, itaconic acid, fumaric acid, crotonic acid, citraconic acid, maleic acid, oleic acid, B-carboxyethyl acrylate, 2-sulfoethyl methacrylate, styrene sulfonic acid, 2-acrylamido-2-methylpropane sulfonic acid, vinyl phosphonic acid, and the like, and mixtures thereof.

Due to their availability, acidic monomers of the present disclosure are typically the ethylenically unsaturated carboxylic acids. When even stronger acids are desired, acidic monomers include the ethylenically unsaturated sulfonic acids and ethylenically unsaturated phosphonic acids. Sulfonic and phosphonic acids generally provide a stronger interaction with a basic polymer. This stronger interaction can lead to greater improvements in cohesive strength, as well as higher temperature resistance and solvent resistance of the adhesive.

Suitable basic monomers for preparing (meth)acrylic PSAs include those containing amine functionality such as vinyl pyridine, N,N-diethylaminoethyl methacrylate, N,N-dimethylamino-ethyl methacrylate, N,N-diethylaminoethyl acrylate, N,N-dimethylaminoethyl acrylate, and N-t-butylaminoethyl methacrylate. Preferred basic monomers include N,N-dimethylaminoethyl methacrylate, and N,N-dimethylaminoethyl acrylate.

The (meth)acrylic PSAs may be self-tacky or tackified. Useful tackifiers for (meth)acrylics are rosin esters such as that available under the trade name FORAL 85 from Hercules, Inc., aromatic resins such as that available under the trade name PICCOTEX LC-55WK from Hercules, Inc., aliphatic resins such as that available under the trade name PICCOTAC 95 from Hercules, Inc., and terpene resins such as that available under the trade names PICCOLYTE A-115 and ZONAREZ B-100 from Arizona Chemical Co. Other materials can be added for special purposes, including hydrogenated butyl rubber, pigments, and curing agents to vulcanize the adhesive partially. Examples of acid-modified tackifiers include acid-modified polyhydric alcohol rosin ester tackifiers as described in U.S. Pat. No. 5,120,781 (Johnson).

In certain embodiments, the (e.g. acrylic) PSA comprises polymerized unit of a poly(alkylene oxide) such as poly(ethylene oxide) and/or poly(propylene oxide). The PSA typically comprises at least 5, 10 or 15 wt.-% and typically no greater than about 30 wt.-% of polymerized poly(alkylene oxide).

In some embodiments, a poly(alkylene oxide) copolymer is blended with a (meth)acrylic copolymer. Examples of useful poly(alkylene oxide) copolymers include, but are not limited to, those poly(alkylene oxide) copolymers available under the trade designations TETRONIC (tetrafunctional block copolymers derived from sequential addition of propylene oxide and ethylene oxide to ethylene diamine with hydrophilic endblocks) and TETRONIC R (tetrafunctional block copolymers derived from sequential addition of propylene oxide and ethylene oxide to ethylene diamine with hydrophobic endblocks) copolymers available from BASF, Mt. Olive, N.J.; PLURONIC (triblock copolymers with poly(ethylene oxide) end blocks and poly(propylene oxide) midblock) and PLURONIC R (triblock copolymers with poly(propylene oxide) endblocks and poly(ethylene oxide) midblock) copolymers available from BASF; UCON Fluids (random copolymers of ethylene oxide and propylene oxide) available from Union Carbide, Danbury, Conn. Various combinations of poly(alkylene oxide) copolymers can also be used. Preferred nonreactive hydrophilic polymer components are block copolymers of polyethylene glycol and propylene glycol available from BASF, Germany under the trade name PLURONIC.

In other embodiments, a poly(alkylene oxide) monomer having a copolymerizable (e.g. vinyl) group is included during the polymerization of the acrylic polymer. Commercially available monomers include 2-(2-ethoxyethoxy)ethyl acrylate which is available under the trade designation “SR-256” from Sartomer Company, West Chester, Pa.; the methoxy poly(ethylene oxide) acrylate which is available under the trade designation “No. 8816” from Monomer-Polymer & Dajac Laboratories, Inc., Trevose, Pa.; the methoxy poly(ethylene oxide) methacrylates of 200 Daltons, 400 Daltons, and 1000 Daltons which are available under the trade designations “No. 16664”, “No. 16665” and “No. 16666”, respectively, from Polysciences, Inc., Warrington, Pa.; and the hydroxy poly(ethylene oxide) methacrylate which is available under the trade designation “No. 16712” from Polysciences, Inc., Warrington, Pa.

Examples of acrylic adhesive compositions include a 97:3 iso-octyl acrylate:acrylamide copolymer 65:15:20 2-ethylhexylacrylate:acrylic acid:copolymer blended with a nonreactive polyakylene oxide copolymer under the trade designation PLURONIC. Other suitable examples include a 90:10 iso-octyl acrylate:acrylic acid copolymer, a 70:15:15 isooctyl acrylate:ethylene oxide acrylate:acrylic acid terpolymer, and a 25:69:6 2-ethylhexylacrylate:butyl acrylate:acrylic acid terpolymer. Additional useful adhesives are described in U.S. Pat. Nos. 3,389,827, 4,112,213, 4,310,509, and 4,323,557.

Inclusion of medicaments or antimicrobial agents in the adhesive is also contemplated, as described in U.S. Pat. Nos. 4,310,509 and 4,323,557.

Pressure sensitive adhesives for wound dressings preferably transmit moisture vapor at a rate greater to or equal to that of human skin. While such a characteristic can be achieved through the selection of an appropriate adhesive, it is also contemplated in the present disclosure that other methods of achieving a high relative rate of moisture vapor transmission may be used, such as pattern coating the adhesive on the backing, as described in U.S. Pat. No. 4,595,001 (Potter et al.).

A composite of flexible film layer coated with pressure-sensitive adhesive layer preferably has a moisture vapor transmission rate of at least 300 g/m²/24 hrs/37° C./100%-10% relative humidity (“RH”), more preferably at least 700 g/m²/24 hrs/37° C./100%-10% RH, and even more preferably at least 2000 g/m²/24 hrs/37° C./100%-10% RH using the inverted cup method as described in U.S. Pat. No. 4,595,001.

In some embodiment, the method of making a fibrin article generally comprises providing a (e.g. dehydrated) fibrin composition and disposing the fibrin composition on or within a carrier. In some embodiments, the carrier is a substrate such as a release liner, a polymeric film or foam, or a nonwoven or woven fibrous material. When the fibrin composition is in a particle form, the methods of making the wound dressing can include distributing fibrin particles onto a (e.g. pressure-sensitive) adhesive layer disposed on a carrier. Alternatively, the fibrin particles can be suspended in a liquid (e.g., an inert, volatile fluorinated liquid) and spray dried in a dehydrated form onto the surface of a (e.g. pressure-sensitive adhesive) layer disposed on a substrate. Examples of suitable wound dressings that include a pressure-sensitive adhesive layer disposed on flexible film layer include TEGADERM wound dressings (e.g., TEGADERM 1626) available from 3M Co., St. Paul, Minn. In one embodiment, the fibrin-containing layer (e.g. sheet or particles) are applied to the surface of a pressure-sensitive adhesive layer of a TEGADERM wound dressing.

A wound dressing article of the present description is typically provided in a package format (i.e., positioned in a sealed package). The interior of the sealed package is typically sterile. Examples of wound dressing packages suitable for use with the wound dressings and methods of this disclosure include, for example, polymeric packages and foil packages. A wide variety of polymeric materials may be used to make non-porous packages suitable for use with the wound dressings. The packaging material may be, for example, polyethylene, polypropylene, copolymers of ethylene and propylene, polybutadiene, ethylene-vinyl acetate, ethylene-acrylic acid, or ionomeric films. Suitable foil packages can include aluminum foil packages. In some embodiments, the packaging material may be used as sheets of material which are placed above and below the wound dressing and then sealed on four sides to generate the package. In other embodiments, a pre-made pouch is utilized which has 3 sides already sealed. After the wound dressing article is placed within the pouch the fourth side is sealed to form the package. Sealing of the package can be achieved by heat sealing (i.e. by the application of heat and pressure to form a seal) or the use of adhesive sealants can be used to seal the packages (for example pressure sensitive adhesive sealants or cold seal sealants). Typically, heat sealing is used. Additionally, packaging systems can be used which include placing the wound dressing in a porous package that is then placed in a non-porous package, such as a foil package. The foil package prevents moisture loss prior to use and the porous package permits easy handling during use.

An advantage of a wound dressing article of the present disclosure is that it can be sterilized by a terminal sterilization process that includes exposure to ethylene oxide or, advantageously, gamma-irradiation. This irradiation can be carried out whether or not the wound dressing article is contained within a package. The exposure times and levels of radiation doses applied to the wound dressings to achieve sterilization can vary based upon a variety of factors, including the gamma equipment used as well as the inherent bioburden levels present in the wound dressing. Typically, to achieve sterilization of a wound dressing, a Sterility Assurance Level (SAL) of 10⁻⁶ is required. This SAL level is typically achieved by exposing the wound dressing to a minimum cumulative gamma irradiation dose. Depending on the bioburden levels in an unsterilized dressing and the size of the dressing, the minimum cumulative dose can range from about 10 kGy to about 35 kGy. Typically the minimum cumulative dose is about 15 to 30 kGy. The required gamma radiation dose to achieve sterility can be done in a single pass or multiple passes through the gamma irradiation sterilizer. For example, exposing the wound dressing to 5 sterilization cycles using a dose of 5 kGy per cycle would be similar to exposing the wound dressing to one dose of 25 kGy of gamma irradiation. Due to labor and time constraints, it is generally desirable to minimize the number of passes that a wound dressing experiences through the gamma irradiation sterilizer. Typically, it is desirable that the number of passes through the sterilizer be five or less, and it may be even more desirable for the number of passes to be two or less. Exposure time may be viewed as the time a sample to be sterilized is exposed to the gamma radiation. Typically the exposure time is on the order of hours.

Gamma radiation is a suitable method to sterilize the wound dressings of this disclosure. Exposure of the wound dressings of this disclosure to a suitable level gamma irradiation does not produce a comparable loss of re-epithelialization performance.

The ability to use terminal sterilization can provide an advantage over other forms of wound dressings that include, for example, a liquid. Without being bound by theory, aqueous solutions or suspensions of proteins such as fibrinogen and thrombin can be expected to undergo inter-chain crosslinking during terminal sterilization that involves gamma-irradiation. In a dry format, a protein will often undergo chain scission (i.e., degradation) and thereby lose enzymatic activity. Thus, gamma-irradiation of the reagents for a polymerization (e.g., fibrinogen and/or thrombin) may result in crosslinking and/or chain scission of the separate reagents, and thus no reaction (or no polymerization) to form fibrin. Depending on the level of gamma-irradiation, fibrin may also undergo some chain scission, although even with low levels of degradation, the gamma-irradiated fibrin still can be recognized by cells to obtain the desired re-epithelialization effect.

The (e.g. dehydrated) fibrin hydrogel in its various physical forms can be utilized for the treatment of wounds. Thus, in another embodiment, a method of treatment of a (e.g. mammal or human) wound is described providing the fibrin composition as described herein or a wound dressing comprising the described fibrin composition and providing the fibrin composition proximate a wound. In typical embodiments, the fibrin-containing layer (e.g. sheet, foam, particles) is in direct contact with at least a portion or portions of the wound. Alternatively, it is surmised that the fibrin-containing layer may be in close proximity, yet not in direct contact. For example, it is contemplated that an absorbent porous substrate, such as a gauze, may comprise the fibrin-containing layer on the opposing surface as the wound facing surface. During use fluids of the wound penetrate through the absorbent porous substrate thereby solubilizing the fibrin-containing layer.

The fibrin composition has been shown to increase the rate of re-epithelialization in both in-vivo porcine studies and in-vitro studies using human primary isolated cells. In some embodiments, the re-epithelialization was 2 times faster than the control (same dressing without (e.g. dehydrated fibrin hydrogel).

The dehydrated fibrin composition was also been found to affect the formation of pro-healing and anti-healing biomarkers such as growth factors, proteases, cytokines as commonly known in the art. (See Murphy, K., Janeway's Immunobiology (E. Lawrence Ed. 8th ed., Garland Science (2012)). In some embodiments, the formation of VEGF—vascular endothelial growth factor was at least 1, 2, 3, or 4 times greater than the control. In some embodiments, the EGF—epidermal growth factor was as least 1 or 2 times greater than the control. In some embodiments, the formation of matrix metalloproteinase—MMP1 and/or MMP8—was at least 1, 2, 3, 4, 5, 6, 7, 8, or 9 times greater than the control. In some embodiments, the formation of matrix metalloproteinase—MMP9—was at least 10, 20, 30, 40, 50, 60, 70, or 80 times greater than the control. In some embodiments, the formation of TIMP1—tissue inhibitor of metalloproteinase was at least 1, 2, 3, or 4 times greater than the control. Prohealing markers IL-4, IL-6, IL-10, EGF, FGF-basic were the same as the control, indicating no effect. Further, anti-healing biomarkers TNF-alpha, IL1-alpha, IL-1beta, IL-2 were all below the detection limit of the assay, indicating a low pro-inflammatory profile.

All patents and patent applications cites herein are incorporated by reference. Other modifications and variations to the present disclosure may be practiced by those of ordinary skill in the art, without departing from the spirit and scope of the present disclosure. It is understood that aspects of the various embodiments may be interchanged in whole or part or combined with other aspects of the various embodiments. The preceding description, given in order to enable one of ordinary skill in the art to practice the claimed disclosure, is not to be construed as limiting the scope of the disclosure, which is defined by the claims and all equivalents thereto.

The operation of various exemplary embodiments of the present disclosure will be further described with regard to the following detailed examples. These examples are offered to further illustrate the various specific and preferred embodiments and techniques. It should be understood, however, that many variations and modifications may be made while remaining within the scope of the present disclosure.

### EXAMPLES

These Examples are merely for illustrative purposes and are not meant to be overly limiting on the scope of the appended claims. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. At the very least, and not as an attempt to limit the application of the doctrine of equivalents to the scope of the claims, each numerical parameter should at least be construed in light of the number of reported significant digits and by applying ordinary rounding techniques.

### Materials

Unless otherwise noted, all parts, percentages, ratios, etc. in the Examples and the rest of the specification are by weight. HEPES, CaCl₂, NaCl, solvents and other reagents used may be obtained from Sigma-Aldrich Chemical Company (Milwaukee, Wis.) unless otherwise noted. Aldrich Chemical Company (Milwaukee, Wis.) unless otherwise noted. Table A provides abbreviations and a source for some common materials used in the Examples below:

TABLE A
Materials

| Ingredient | Description |
|---|---|
| Fibrinogen | Bovogen Biologicals (Keilor East VIC, Australia) |
| Thrombin | MP Biomedicals (Seven Hills NSW, Australia) |
| Blood Bank Grade | Azer Scientific (Morgantown, PA) |
| Saline | |
| ACS Grade Calcium | BDH Chemicals Limited (Mumbai, India) |
| Chloride | |
| (CaCl₂) | |
| Bioreagent Grade | Sigma-Aldrich (St. Louis, MO) |
| Glycerol | |

### Preparative Examples—Formation of a Fibrin Gel Composition

Fibrinogen was converted to a fibrin gel dispersion as follows. Lyophilized fibrinogen was dissolved in either water or saline. In some formulations calcium chloride was included. In formulations in which calcium chloride was included it was added to the aqueous solution after the fibrinogen was dissolved. A solution of thrombin, a molecule with enzymatic activity, was then added to the fibrinogen solution. The amount of thrombin added to the formulation was recorded in units of enzymatic activity per mL. After the addition of the thrombin the solution was stirred no longer than one minute. A fibrin gel formed in less than an hour. The fibrin gel was then allowed to sit at room temperature for a minimum of 12 hours in order to achieve more complete conversion of the fibrinogen to the fibrin gel.

Table B summarizes the different fibrin gels that were prepared from different fibrinogen solutions. The solvent and amounts of each raw material used in the preparation of different fibrin gels. The percentages listed are in w/w based on the amount of fibrinogen solution.

TABLE B
Preparative Fibrin Gel Examples

| Preparative | | Fibrinogen | CaCl₂ | Thrombin |
|---|---|---|---|---|
| Example | Solvent | (%) | (%) | (units/nip |
| 1 | Saline | 2 | 0.3 | 0.56 |
| 2 | Saline | 4 | 0.3 | 0.44 |
| 3 | Saline | 4 | 0.3 | 0.57 |
| 4 | Saline | 6 | 0.3 | 0.55 |
| 5 | Saline | 10 | 0.3 | 0.55 |
| 6 | Water | 4 | 0 | 0.55 |

Transformation of the Fibrin Gels into an Aqueous Fibrin Gel Dispersion

After twelve hours the fibrin gels were broken up into gel pieces having a long dimension of 1-10 cm using a laboratory spatula. The gel pieces were then sheared or chopped using a Model L5M-A laboratory Mixer-Homogenizer commercially available from Silverson Machines, Inc, (East Longmeadow, Mass.), at rotational speeds that did not exceed 6,000 rpm. The use of this apparatus required that a small amount of water be added to the fibrin gels of Examples 2-6 to ensure that the mixer head was submerged in the solution during the homogenizing process. The gel pieces were sheared or chopped until the fibrin gel pieces were substantially uniformly sized.

After the fibrin gel samples were sheared or chopped, some of them were washed to remove residual salt and to concentrate the fibrin gel. In this washing, the chopped fibrin gel pieces were first placed in a bag or pocket constructed of a wear resistant nylon mesh, 104×104 with 0.0059″ openings, commercially available from McMaster Can (Elmhurst, Ill.). The fibrin gel pieces in the bag or pocket were then soaked in water, the volume of water used being equivalent to the amount of solvent used to initially dissolve the fibrinogen. The excess water was then allowed to drain from the fibrin gel pieces through the nylon mesh. The fibrin gel pieces were then removed from the water and the excess water was allowed to drain. This process was repeated until the fibrin gel pieces had been washed a total of three times.

Glycerol and optionally water was then added to the washed chopped fibrin gel pieces to form a fibrin gel dispersion in an aqueous medium, which was then mixed by stirring. A minimum of 4% w/w glycerol was added to the fibrin gel pieces, based on the weight of the fibrin gel dispersion.

A dye, commercially available as FD&C Red #40 from Sensient (Milwaukee, Wis.) was added to some of the fibrin gel dispersions. The dye was intended to aid in process optimization of the fibrin gel film-making process by enabling the fibrin gel film to be seen through the nonwoven webs during the dewatering process that will be discussed below.

Each fibrin gel dispersion was then analyzed in a Sartorius Moisture Analyzer, commercially available from Sartorius (Göttingen, Germany) to determine the percent solids. Table C shows which samples were and were not washed, which had a dye added, and the percent solids present in each fibrin gel dispersion.

TABLE C
Fibrin Gel Aqueous Dispersion Examples

| | | | Solids in | |
|---|---|---|---|---|
| Aqueous | | | Fibrin Gel | Dye |
| Dispersion | | Washed | Dispersion | Added |
| Examples | Solvent | (yes/no) | (%) | (yes/no) |
| 1 | Saline | No | 2.35% | Yes |
| 2 | Saline | No | 11.92% | Yes |
| 3 | Saline | Yes | 8.51% | No |
| 4 | Saline | Yes | 10.1% | No |
| 5 | Saline | Yes | NA | No |
| 6A | Water | No | 4.68% | No |
| 6B | Water | Yes | 15.2% | No |

Consolidating the Aqueous Dispersion into a Cohesive Substrate

An apparatus generally as described in connection with FIG. 1 was constructed. The several fibrin gel dispersions in the aqueous medium described in Table 3 were then supplied in separate runs to the trough. In these experiments, both the scrim and the carrier substrate were an indefinite length substrate of an SMS oriented polypropylene nonwoven web commercially available as UNIPRO 150 from Midwest Filtration LLC (Cincinnati, Ohio).

The surfaces of the nip roller and the backup roller at the dispensing station were formed firm but resilient polymer, and the nip was set at a fixed gap of 1.6 mm. The multi-layer substrate emerging from the dispensing station was conveyed at a line speed of 5 feet/min (1.52 m/min) was gradually dewatered by passing the material through two wringing stations having nips of decreasing clearance. The surfaces of the nip roller and the backup roller at the first wringing station were formed firm but resilient polymer, and the nip was set at a fixed gap of 75 mil (1.91 mm). The multi-layer substrate was then conveyed to a second wringing station. The surfaces of the nip roller and the backup roller at the second wringing station were formed firm but resilient polymer, and the nip was set at a fixed gap of 40 mil (1.02 mm). The multi-layer substrate was then conveyed to the consolidating station where rather than a fixed gap, the nip provided a controlled pressure of 90 psi (0.62 MPa).

The multi-layer substrates were then conveyed into a forced air drying oven where the air temperature was maintained at 267° F. (130° C.). Only a 10 foot oven was available for the experiments, and in some Examples the planned time in the oven was insufficient to effect complete drying. Stopping the line briefly while samples to be tested were in the oven was used as an expedient expected to be unneeded in circumstances where a longer oven is available.

Once the multi-layer substrates were completely dry, the scrim was stripped away and the consolidated and the dried fibrin material was wound on the windup roller. It was found that the Aqueous Dispersion Examples 1 and 5 did not form a continuous substrate that could be successfully wound and thereafter unwound such that the fibrin substrate could be peeled whole from the carrier substrate. In particular, in the case of Aqueous Dispersion Example 5, discrete flakes of fibrin were formed.

The fibrin substrate from Example 3 was peeled from the carrier substrate was cut into 95 individual pieces, each 3.25 inch×2.25 inch (8.26 cm×5.72 cm). The pieces were weighted to determine how uniform the basis weight of the fibrin film was in different locations. The basis weight of the film was 6.8 mg/cm²±0.03 (standard deviation).

### In-Vivo Testing of Fibrin Gel Sheets

In-vivo testing of the fibrin gel sheet reproduced using Aqueous Dispersion Example 4 consisted of a 6-pig study with a 72-hr endpoint, partial-thickness wound studies using a porcine model. There were 6 wounds per pig, each 5 cm×7.6 cm (2×3 inch) in area and 500 micrometers deep. Testing was conducted using an IACUC approved protocol and care was taken to ensure proper animal treatment and minimize unnecessary pain.

On the day of wound creation, the wound area was shaved and prepped for sterile surgery. Wound areas were marked with a sterile marker and sterile mineral oil was placed over the wound area to facilitate the dermatome procedure. After wound creation, absorbent gauze was applied with light pressure for 5 minutes to achieve hemostasis. The wound margins were then painted with a benzoin tincture to improve adherence of adhesive bandages. Wounds were treated either with the fibrin gel sheet of Example 4 and then covered with 3M TEGADERM HP FOAM DRESSING, available from 3M Company (St. Paul, Minn.) under 3M Catalog Number 90601,) or controls were covered with only the 3M TEGADERM HP FOAM DRESSING (no fibrin gel sheet), considered a standard of care for wounds. When individual dressings were in place, the edges were taped down using 3M 1363 Veterinary Elastic Adhesive Tape, available from 3M Company (St. Paul, Minn.). Then all wounds were covered with an organza cloth overlay.

At the conclusion of the study, the animal was euthanized according to approved protocols. Tissue samples were then collected for histology and biochemical analysis. Histology samples were placed into 10% neutral buffered formalin (available from Thermo Scientific, Minneapolis, Minn.) for fixation. Samples were then prepped for paraffin embedding, microtomed to 6 micrometers sections and stained with hematoxylin and eosin (H&E).

The H&E samples were analyzed for percent re-epithelialization by measuring the width of the wound covered with keratinocytes and dividing that value by the measured width of the wound. Wounds treated with the fibrin gel sheet made from a plurality of pieces of Example X (made with from an initial fibirinogen concentration of 0.9% w/w, 1000 units of thrombin) exhibited 41.1±7.0% re-epithelialization rate and the foam dressing control exhibited a re-epithelialization rate of 29.6±5.1% (mean±95% confidence interval, n=6). Thus, treatment with a lyophilized fibrin gel sheet made from a plurality of pieces of Example 4, resulted in approximately 1.5 times faster re-epithelialization compared to the standard of care (the foam dressing control).

### Preparation of Fibrin Gel Sheets with Various Salt Concentrations

### Examples 1-3 and Comparative Examples C1-C6

Various fibrin gel dispersions were cast into sheets. Fibrin gels were prepared by dissolving 2.7 g fibrinogen (SIGMA catalog number F8630, available from SIGMA-Aldrich (Milwaukee, Wis.) in 592.5 mL water with various salts as shown in Table 1 below, plus 1% w/w glycerol (SIGMA catalog number G2025). Next, 0.6 Units of thrombin (SIGMA catalog number T7009) per mg of fibrinogen was added to the fibrinogen solution to initiate polymerization. This solution mixture was mixed for 20-30 seconds and then transferred to a 6-well plate to finalize the polymerization. The mixture was incubated at 37° C. for 30 minutes and then evaluated qualitatively for gel formation, as indicated by visual observation of a continuous opaque white material formed in the well of the plate that could be removed from the plate as a single mass.

If the components did not form a gel, one of two failure modes was recorded. In the case of Failure Mode 1, precipitation of components was observed and the precipitated solids were surrounded by unpolymerized aqueous solution. In the case of Failure Mode 2, no differences were observed before and after combination of the components. Thus, the composition remained an aqueous ungelled solution. The post-drying salt content was determined by measuring the conductivity of a solution containing 1% w/w of a fibrin sheet dehydrated to a water content of about 10% in 18.2 megohm-cm at 25° C. water.

The moisture content was determined by calculating the weight loss of a (completely) dehydrated sample of the same film. The (completely) dehydrated sample was conditioned in a convection oven at 60° C. for 24 hours.

TABLE 1
Composition, Salt Content and Gel formation

| | NaCl | CaCl₂ | NaCl + CaCl₂ | | Post-drying |
|---|---|---|---|---|---|
| | (wt.-% | (wt.-% | (Total wt.-% | Gel | salt content |
| EXAMPLE | solution) | solution) | solution) | formation? | (wt-%) |
| EX.1 | 0.9 | 0.333 | 1.233 | Yes | 41.89 |
| EX.2 | 0.9 | 0.033 | 0.933 | Yes | 35.31 |
| EX.3 | 0.9 | 0.003 | 0.903 | Yes | 34.57 |
| C1 | 0.09 | 0.333 | 0.423 | No (1) | 19.83 |
| C2 | 0 | 0.333 | 0.333 | No (1) | 16.30 |
| C3 | 0.09 | 0.033 | 0.123 | No (2) | 6.73 |
| C4 | 0.09 | 0.003 | 0.093 | No (2) | 5.18 |
| C5 | 0 | 0.033 | 0.033 | No (2) | 1.91 |
| C6 | 0 | 0.003 | 0.003 | No (2) | 0.19 |

### Preparation of Fibrin Gel Sheets for In-Vivo Studies

### Example 4

Fibrin gels were cast by dissolving 2.7 g fibrinogen (SIGMA catalog number F8630) in 592.5 mL of 20 mM HEPES, pH 7.4 (AMRESCO catalog number 0511) in 0.9% NaCl, plus 1% w/w glycerol (SIGMA catalog number G2025). To this solution, 2.0 g CaCl₂ (SIGMA catalog number C5670) was added. Next, 0.06 Units of thrombin (SIGMA catalog number T7000) per mg of fibrinogen (resulting in a thrombrin concentration of 0.27 U/mL) was added to the fibrinogen solution to initiate polymerization. This solution was mixed for 20-30 seconds and then cast into a lyophilizer pan resulting in a gel that was approximately 7 mm thick. The gel was incubated at 37° C. for 30-60 minutes. The fibrin hydrogel prior to dehydration had a fibrin content of about 0.45 wt.-%, a salt content of about 0.6 wt. %, a glycerol content of about 1%, and the remainder water (about 98%).

The fibrin hydrogel was then placed into a solution of ultra-pure water (18.2 megohm-cm at 25° C.) and 1% w/w glycerol. The volume of this solution was 10 times greater than the volume of the gel. The gel was rinsed in this solution overnight, then placed back into the lyophilizer pan from which it was cast. The gel was then freeze-dried using standard methods. The resulting sheet was a flexible fibrin gel sheet with a thickness of approximately 50 micrometers. The resulting rinsed dehydrated film of fibrin gel had a fibrin content of about 50 wt.-%, a glycerol content of about 30 wt.-% and a water content of about 10 wt.-%. The resulting rinsed dehydrated film of fibrin gel sheet was found to have a salt content of approximately 10%, as determined by measuring the conductivity of a solution containing 1% w/w of the fibrin sheet in 18.2 megohm-cm at 25° C. water. The sheet was then cut into 5 cm×7.6 cm sections to be used for in vivo, partial-thickness wound studies using a porcine model.

### Example 4: In-Vivo Testing Protocol

In-vivo testing of the fibrin gel sheet consisted of a 6-pig study with a 72-hr endpoint, partial-thickness wound studies using a porcine model. There were 6 wounds per pig, each 5 cm×7.6 cm (2×3 inch) in area and 500 micrometers deep. Testing was conducted using an IACUC approved protocol and care was taken to ensure proper animal treatment and minimize unnecessary pain.

On the day of wound creation, the wound area was shaved and prepped for sterile surgery. Wound areas were marked with a sterile marker and sterile mineral oil was placed over the wound area to facilitate the dermatome procedure. After wound creation, absorbent gauze was applied with light pressure for 5 minutes to achieve hemostasis. The wound margins were then painted with a benzoin tincture to improve adherence of adhesive bandages. Wounds were treated either with the fibrin gel sheet of Example 4 and then covered with 3M TEGADERM HP FOAM DRESSING (3M catalog number 90601) or controls were covered with only the 3M TEGADERM HP FOAM DRESSING (no fibrin gel sheet), considered a standard of care for wounds. When individual dressings were in place, the edges were taped down using 3M 1363 Veterinary Elastic Adhesive Tape. Then all wounds were covered with an organza cloth overlay.

At the conclusion of the study, the animal was euthanized according to approved protocols. Tissue samples were then collected for histology and biochemical analysis. Histology samples were placed into 10% neutral buffered formalin (Thermo Scientific catalog number 534801) for fixation. Samples were then prepped for paraffin embedding, microtomed to 6 micrometers sections and stained with hematoxylin and eosin (H&E).

### Example 4: In-Vivo Testing Results

The H&E samples were analyzed for percent re-epithelialization by measuring the width of the wound covered with keratinocytes and dividing that value by the measured width of the wound. Wounds treated with the fibrin gel sheet of Example 4 exhibited 49.8±4.9% re-epithelialization rate and the foam dressing control exhibited a re-epithelialization rate of 23.8±4.1% (mean±95% confidence interval, n=16). The conclusion was that treatment with a lyophilized fibrin gel sheet of Example 4, resulted in approximately 2 times faster re-epithelialization compared to standard of care (the foam dressing control).

### Example 4: Biochemical Indications of Wound Healing

In addition to percent re-epithelialization, tissue samples of wounds treated with Example 4 and the foam dressing control were also analyzed for biomarkers. Wounds were biopsied following the fibrin gel sheet treatment for 72 hr. Wound biopsies were homogenized with a blender and analyzed for wound healing and inflammatory biomarkers. A multiplex ELISA assay was used to determine pro-healing and anti-healing wound outcomes. Selected screening panel included the following: (A) Pro-healing biomarkers: IL-4, IL-6, IL-10, EGF, FGF-basic, VEGF, MMP-1, MMP-3, MMP-8, MMP-9, TIMP-1; and (B) Anti-healing biomarkers: TNF-alpha, IL1-alpha, IL-1beta, IL-2. The data shown in TABLE 2 are representative of 4 animals with 2 biopsies of 2 wounds for each treatment. TABLE 2 shows results for the biomarkers of wound healing, summarized as X-fold increase over the control (3M TEGADERM HP FOAM DRESSING) in Example 4.

The results indicate significant changes observed with fibrin treatment compared to the standard of care (control), as well as trends (greater than 5 fold up-regulation) of fibrin mediated biomarker induction, indicative of wound healing. The anti-healing biomarkers tested were all below the detection limit of the assay (data not shown), indicating a low pro-inflammatory profile and further confirming the capability of the fibrin gel sheet of Example 4 to accelerate the wound healing process toward completion. Statistical significance was determined via student's t-test where significance was determined at p<0.05.

TABLE 2
Example 4—Wound Healing Biomarker Analysis

| | X-fold Change in Up-Regulation | |
|---|---|---|
| | Example 4 Fibrin Treatment vs. Control | |
| Biomarker | Average ± std. dev. | p-value |
| VEGF | 4.3 ± 0.9 | 0.0005 |
| EGF | 2.1 ± 0.03 | 0.0005 |
| MMP1 | 8.9 ± 11.9 | ns |
| MMP8 | 6.4 ± 4.9 | 0.034 |
| MMP9 | 75.6 ± 4.63 | ns |
| TIMP-1 | 3.45 ± 2.05 | 0.019 |

### Example 4: Mechanical Testing Results

The fibrin gel sheets of Example 4 were tested for mechanical properties using an INSTRON Tensile Tester Model 5943 with a 5 kg-force load cell. The dried (lyophilized) gel sheets of Example 4 was cut to a width of 6.2 mm. Thickness of the gel sheets was measured by micrometer to determine cross-sectional area of tested samples. The tensile testing apparatus was calibrated for grip spacing at each measurement. Samples were mounted between tensile grip adapters and elongated at a rate of 50 mm/min. Data acquisition was triggered at 0.02 N of applied force. Resulting strain was calculated in situ using the cross-sectional area defined by the input sample measurements for each test. Young's Modulus was calculated from the linear region of the stress-strain curve and defined as between 0.2% and 2% strain.

TABLE 3
Example 4—Mechanical Testing Results

| | Ultimate | Young's | | | |
|---|---|---|---|---|---|
| | Tensile | Elastic | | | Basis |
| | Strength | Modulus | % | Thickness | Weight |
| Example 4 | (MPa) | (MPa) | Elongation | (microns) | (mg/cm²) |
| Dried fibrin | 3.41 +/− | 32.9 +/− | 75.2 +/− | 44.7 ± 1.9 | 4.12 |
| gel sheet | 0.79 | 2.53 | 26.7 | | |

### Alternative Drying Methods for Fibrin Gel Sheet Preparation

### Example 5

Fibrin gel sheets were prepared by the same method described in Example 4. The only changes were (1) the source of fibrinogen and thrombin were both obtained from Cambryn Biologics LLC, of Sarasota, Fla. and (2) two different drying techniques were evaluated. Fibrin gel sheet samples were dried using (i) the lyophilization method as outlined above or (ii) dried in a convection oven at 60° C. for 3-5 hours. The moisture content of both films was 10% or less.

The purpose of this example was to compare oven drying to lyophilization as a method to dehydrate the fibrin gels. Oven drying of proteins is not generally an acceptable process because tertiary structure of proteins is easily lost when heated.

The oven-dried fibrin gel sheets were observed to be more transparent and more uniform than the lyophilized sheets. The lyophilized fibrin gel sheets, though similar in composition, were more opaque due to the formation of ice crystals within the sheet as part of this dehydration process. Also the fibrin gel sheet s dried by lyophilization exhibited a more random variation in opacity.

### Example 5: In-Vivo Testing Protocol

The in-vivo testing of the fibrin gel sheets of Example 5 was done similarly to Example 4. A 2-pig study was conducted with a 72 hour endpoint. Other than treatment groups (lyophilized fibrin gel sheet, oven-dried fibrin gel sheet, or control—3M foam dressing only), there were no differences in the protocol compared to that which was performed in Example 4.

### Example 5: In-Vivo Testing Results

H&E samples were analyzed for percent re-epithelialization by measuring the width of the wound covered with keratinocytes and dividing that value by the measured width of the wound as was done in Example 4. A summary of the percent re-epithelialization results for the in-vivo testing of Example 5 samples (mean±SEM, n=4 per treatment) are shown below in TABLE 4. On average, fibrin gel sheet treatments of Example 5 exhibited 2 times more re-epithelialized than the control group (3M foam dressing only). There was no statistical difference between drying methods regarding re-epithelialization.

TABLE 4
EXAMPLE 5—Percent Re-epithelialization Results

| Wound Treatment: Drying Method | % Re-epithelialization |
|---|---|
| Control (3M Foam Dressing) | 29.4 ± 7.4% |
| Example 5: Oven-dried Sheet | 61.1 ± 7.3% |
| Example 5: Lyophilized Sheet | 52.9 ± 9.7% |

### Comparative Example C7

Comparative Example C7 was prepared to demonstrate the impact of insufficient washing on fibrin formation and the implications for wound healing, without glycerol present.

Preparation of a fibrin gel powder. A fibrin gel was first prepared using the same procedure as Example 4 with the following exceptions. The thrombin was sourced from SIGMA-Aldrich, cat. No. T6634. Also there was no glycerol added and the resulting gel was not washed. The solution for polymerization of a fibrin gel was prepared in a 50 mL centrifuge tube. The polymerized gel was lyophilized by freezing to −40° C., followed by pulling a vacuum to approximately 500 mTorr and ramping the temperature up to 20° C. while maintaining vacuum. The dried gel was then crushed into a powder by mortar and pestle.

A pressure sensitive adhesive (PSA) solution (isooctyl acrylate and acrylamide combined in a 97:3 weight ratio and dissolved at 33 wt. % solids in a solvent mixture of 51 wt. % heptane and 49 wt. % ethyl acetate (EtOAc)) was diluted 1:1 by volume in pentane. This solution was then put into an aerosolization jar, and then was sprayed onto a layer of an absorbent foam (obtained from 3M Co., St. Paul, Minn., under the trade designation “3M 90600 TEGADERM FOAM DRESSING (NONADHESIVE)”), followed by drying at 50° C. for 10 minutes, to provide an adhesive coating weight of 11.5 mg/cm² on the absorbent foam. The fibrin powder (described above—unwashed and without glycerol) was dry “shaker” coated onto the resulting adhesive coated surface with a resulting fibrin powder coating weight of 3.7 mg/cm².

The gel was not washed to remove any salts; thus, the resulting dried material was 65.5% w/w salt, 28.9% fibrin and the balance water. Salt content was determined by measuring the conductivity of a solution containing 1% w/w fibrin sheet material in 18.2 megohm-cm at 25° C. water.

### Comparative Example C7: In-Vivo Testing Results

An in-vivo porcine study following the protocol set out in Example 4 was performed using the Comparative Example C7. The wound tissue treated with Comparative Example C7 was found to be highly irritated. Histology further demonstrated signs of dermal damage during the healing process in the presence of this high salt-content material, evidenced by the presence of high numbers of neutrophils throughout the dermis and collagen degradation and mineralization. As mentioned above histology sections of tissues from Examples 6 and 7 (above) did not demonstrate this effect.

### Example 6 and Comparative Example C8

Example 6 and Comparative Example C8 were prepared to demonstrate the impact of insufficient washing on fibrin formation and the implications for wound healing, with glycerol present. The fibrinogen and thrombin were both obtained from Cambryn Biologics, LLC for these examples. One gel was prepared as in Example 4, but was washed twice with a volume of 18.2 megaohm-cm water with 1% glycerol that was 10 times greater than the original volume of the gel. The other gel was prepared by doubling the formulation listed in Example 4 and washing once with a volume of 18.2 megaohm-cm water with 1% glycerol that was 10 times greater than the original volume of the gel. Salt content of the prepared examples was determined by measuring the conductivity of a solution containing 1% w/w fibrin sheet material in 18.2 megohm-cm at 25° C. water. The glycerol content was determined in the finished fibrin gel sheets by liquid chromatography-mass spectrometry (LC/MS).

The fibrin gel sheet of Example 6 was prepared with extensive washing to reduce the salt concentration to 0.2% w/w and the glycerol content was 33% w/w.

The fibrin gel sheet of Comparative Example C8 was intentionally not sufficiently washed, resulting in a gel that had a salt concentration of 23.6% and a glycerol content of approximately 50%.

### Example 6 and Comparative Example C8: In-Vivo Testing Results

The gel sheets of Example 6 and Comparative Example C8 were evaluated in an in-vivo porcine study following the protocol set out in Example 4. Examination of the wounds for re-epithelialization after 3 days demonstrated that Example 6 promoted wound healing as evidenced by regions of low inflammation and coloration indicative of re-epithelialization. Histological examination of wounds treated with Example 6 showed signs of increased keratinocyte migration over the wound space. The porcine wounds treated with Comparative Example C8 showed signs of tissue damage and necrosis; the wound space became brown/black in color. Histological examination of wounds treated with Comparative Example C8 also showed apoptotic cells, cellular debris, collagen degeneration and vascular necrosis. Quantification of re-epithelialization showed that the wounds treated with Example 6 (washed formulation with lower salt content) healed approximately 2 times faster than the 3M 90600 TEGADERM FOAM DRESSING control.

### Alternative Plasticizing Components Other than Glycerol

### Example 7

Fibrin gels were cast by dissolving 0.54 g fibrinogen (SIGMA-Aldrich, Cat. No. F8630) in 60 mL 20 mM HEPES buffered saline (pH 7.4) to make a stock solution. Mixtures of fibrinogen with different plasticizers were then made by adding 2% w/w plasticizer (TABLE 5) to 5 mL of the stock solution. An amount of 0.4 g CaCl₂) (SIGMA-Aldrich, Cat. No. C5670) was added to a solution of 1.2 U/mL thrombin. Polymerization was initiated by adding equal parts of the fibrinogen and thrombin solutions. The resulting solution was mixed for 20-30 seconds and then cast into a single well of a 6-well plate. The gel was incubated at 37° C. for 30-60 minutes and then placed into a solution of water (18.2 megohm-cm at 25° C.)+1% w/w plasticizer. The volume of this solution was 10 times greater than the volume of the gel. The gel was rinsed in this solution overnight, then placed into a 60° C. oven until dry. All of the plasticizers tested resulted in a flexible fibrin sheet.

TABLE 5
Alternative Plasticizers

| Plasticizer | Supplier | Catalog Number |
|---|---|---|
| 1,3 Butanediol | TCI | B0681 |
| 1,4 Butanediol | Alfa Aesar | A11684 |
| 2,3 Butanediol | Baker Chemical | D570-07 |
| 1,2 Propanediol | Alfa Aesar | 30948 |
| D-mannitol | Alfa Aesar | 33342 |
| Xylitol | Alfa Aesar | A16944 |
| Diglycerol | Solvay | |
| Polyglycerol-3 | Solvay | |

### Example 8

Example 8 was prepared to evaluate a fibrin gel sheet prepared with no glycerol but with adequate rinsing to reduce the salt content in the final article. Sheets were prepared for testing similarly to Example 4 with changes only in the glycerol content of the wash step. Sheets were prepared with both 0% and 1% glycerol in the wash water. After this salt removal step, samples were dried in a convection oven at 60° C. until the moisture content was 10% or less, typically achieved in 3-5 hours. Fibrin preparations that were free of plasticizer were broken into smaller, random sized flakes, typically ranging from 1 cm to about 0.1 mm. Most particles were approximately 0.5 cm to 1 cm, though they were irregularly shaped rather than controlled to a specific shape, e.g. disks, squares or the like.

### Example 8: In-Vivo Testing Results

Example 8 samples were evaluated in an in-vivo (1 pig) porcine study following the protocol set out in Example 4, with a 72 hour endpoint. Other than treatment groups, there were no differences in the protocol compared to that which was shown in Example 4. Treatment groups consisted of fibrin flakes (large pieces of fibrin sheet without plasticizer), flexible fibrin gel sheets and 3M TEGADERM Foam dressing in Example 4.

H&E samples were analyzed for percent re-epithelialization by measuring the width of the wound covered with keratinocytes and dividing that value by the measured width of the wound as performed in Example 4. A summary of the percent re-epithelialization results for the in-vivo testing of Example 8 samples (mean±SEM, n=4 per treatment) are shown below in TABLE 6.

TABLE 6
EXAMPLE 8: Percent Re-epithelialization Results

| Wound Treatment | % Re-epithelialization |
|---|---|
| Control 1 (3M Foam Dressing only) | 36.7 ± 2.3% |
| Example 8: Flexible Fibrin Gel Sheet | 57.5 ± 5.7% |
| (with glycerol) | |
| Example 8: Fibrin Flakes | 70.6 ± 1.4% |
| (no glycerol, no plasticizer) | |

### Foamed Fibrin Article

### Example 9

A foamed fibrin article was prepared in the following manner. Fibrin gel was prepared by preparing a fibrinogen solution as in Example 4. Immediately after the addition of thrombin to initiate polymerization of fibrin, the solution was vigorously mixed for 20-30 seconds so as to aerate the solution. The resulting aerated solution was transferred to a pan to finalize the polymerization. The foam was incubated at 37° C. for 30 minutes and then placed into a solution of 18.2 megohm-cm water+1% w/w glycerol. The volume of this solution was 10 times greater than the volume of the original fibrinogen solution. As in Example 5, the foam was rinsed overnight, then transferred back into the pan from which it was cast. The foam was then be freeze-dried as in Example 4. The resulting dried foam is flexible and has a salt concentration less than 5%.

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment” or “in an embodiment” in various places throughout this specification are not necessarily referring to the same embodiment of the certain exemplary embodiments of the present disclosure. Furthermore, the particular features, structures, materials, or characteristics may be combined in any suitable manner in one or more embodiments.

While the specification has described in detail certain exemplary embodiments, it will be appreciated that those skilled in the art, upon attaining an understanding of the foregoing, may readily conceive of alterations to, variations of, and equivalents to these embodiments. Accordingly, it should be understood that this disclosure is not to be unduly limited to the illustrative embodiments set forth hereinabove.

Furthermore, all publications and patents referenced herein are incorporated by reference in their entirety to the same extent as if each individual publication or patent was specifically and individually indicated to be incorporated by reference. Various exemplary embodiments have been described. These and other embodiments are within the scope of the following claims.

1. A method of forming a fibrin hydrogel composition comprising
providing one or more unitary masses of a fibrin hydrogel comprising fibrin;
dividing at least one of the unitary masses of the fibrin hydrogel into a plurality of smaller pieces of the fibrin hydrogel;
recombining at least a portion of the smaller pieces into a cohesive mass, optionally wherein the cohesive mass is on a substrate.

2. The method of claim 1, wherein dividing at least one of the unitary masses of the fibrin hydrogel into a plurality of smaller pieces of the fibrin hydrogel comprises shearing the one or more unitary masses of the fibrin hydrogel to form an aqueous dispersion of the smaller pieces of the fibrin hydrogel in an aqueous medium.

3. The method of claim 2, further comprising adding an aqueous liquid to the one or more unitary masses of the fibrin hydrogel.

4. The method of claim 1, wherein the smaller pieces of the fibrin hydrogel exhibit a particle size of no greater than 5 mm.

5. The method of claim 3, wherein the one or more unitary masses of the fibrin hydrogel comprise a fibrin hydrogel-forming salt, further wherein the hydrogel-forming salt has a concentration greater than or equal to a threshold concentration required to form a fibrin hydrogel.

6. The method of claim 5, further comprising reducing the concentration of the hydrogel-forming salt below the threshold concentration required to form a fibrin hydrogel.

7. The method of claim 5, wherein the fibrin hydrogel-forming salt is a calcium salt.

8. The method of claim 1, further comprising combining the smaller pieces of the fibrin hydrogel with at least one of a fibrin hydrogel plasticizer, a fibrin hydrogel swelling agent, a water soluble (co)polymer having a Fikentscher K-value of at least K-90, or a combination thereof.

9. The method of claim 2, further comprising casting the aqueous dispersion of the smaller pieces of the fibrin hydrogel in the aqueous medium on a roller or an endless belt, and removing at least a portion of the aqueous medium from the aqueous dispersion, the fibrin hydrogel, or both the aqueous dispersion and the fibrin hydrogel, to form the cohesive mass.

10. The method of claim 9, further comprising providing a carrier substrate between the cast aqueous dispersion and the roller or endless belt.

11. The method of claim 9, wherein casting the aqueous dispersion of the smaller pieces of the fibrin hydrogel in the aqueous medium on the roller or endless belt produces a continuous coating of the fibrin hydrogel on the carrier substrate, or produces a discontinuous coating of the fibrin hydrogel on the carrier substrate.

12. The method of claim 11, wherein the discontinuous coating comprises a plurality of wavy lines, a plurality of parallel lines, a plurality of non-parallel lines, a plurality of dots, or a combination thereof.

13. The method of claim 9, further comprising providing a carrier layer or scrim on a major surface of the cast aqueous dispersion on the carrier substrate.

14. The method of claim 13, wherein removing at least a portion of the dispersion medium from the aqueous dispersion, the fibrin hydrogel, or both the aqueous dispersion and the fibrin hydrogel, comprises applying pressure to the cast aqueous dispersion to form the cohesive mass, optionally wherein applying pressure to the cast aqueous dispersion comprises conveying the cast aqueous dispersion through one or more nip rollers.

15. The method of claim 14, wherein applying pressure to the cast aqueous dispersion comprises at least one of wrapping the cast aqueous dispersion positioned between the carrier substrate and the substrate around one or more rollers while maintaining the carrier substrate and the substrate under tension, wrapping the cast aqueous dispersion positioned between the carrier substrate and the substrate around a water permeable roller while maintaining the carrier substrate and the substrate under tension, or wrapping the cast aqueous dispersion on one or both of the carrier substrate and the substrate around a water permeable roller while maintaining an interior portion of the water permeable roller under at a pressure below atmospheric pressure, and while maintaining the carrier substrate and the substrate under tension.

16. The method of claim 9, wherein removing at least a portion of the aqueous medium from the aqueous dispersion, the fibrin hydrogel, or both the aqueous dispersion and the fibrin hydrogel, comprises heating the cohesive mass, freeze-drying the cohesive mass, vacuum-drying the cohesive mass, contacting the substrate on a side opposite the cast aqueous dispersion with an absorbent material, contacting the cohesive mass with an absorbent material, or a combination thereof.
